(19) United States
(12) Patent Application Publication
(10) Pub. No.: US 2015/0073154 A1
(43) Pub. Date: Mar. 12, 2015

(54) Discrete PEG Based Dyes

(71) Applicant: EquIP, LLC, Plain City, OH (US)

(72) Inventor: Paul D. Davis, Dublin, OH (US)

(73) Assignee: EQUIP, LLC, Plain City, OH (US)

(21) Appl. No.: 14/482,174

(22) Filed: Sep. 10, 2014

Related U.S. Application Data
(60) Provisional application No. 61/876,505, filed on Sep. 11, 2013.

(51) Int. Cl. C07D 209/58; C07D 403/12

(52) U.S. Cl. CPC C07D 209/58; C07D 403/12
USPC 548/427

(57) ABSTRACT

Disclosed are discrete PEGylated dyes, that is, dyes, generally ones that are fluorescent, but could also include chemi-luminescent or electrochemiluminescent and related dye or dye precursors, that have discrete PEG constructs chemically attached in various configurations on the dye, and in the entire range of constructs, discrete PEG compounds (polyethylene glycol oligomers that are made synthetically according to methods disclosed in U.S. Pat. No. 7,888,536 and US Pub. No. 2013/0052130). The dyes are modified in a range of ways to control or optimize the properties of water solubility, non-specific binding (in vitro), biodistribution (in vivo), cell internalization (non-cell or cell based assays in vitro, and in vivo diagnostics and therapy), as well as aggregation.

### CROSS-REFERENCE TO RELATED APPLICATIONS

This application claims benefit of provisional application Ser. No. 61/876,505 filed on Sep. 11, 2013.

### STATEMENT REGARDING FEDERALLY SPONSORED RESEARCH

Not applicable.

### BACKGROUND

The present disclosure relates to dyes and more particularly to those containing primarily discrete PEG constructs to control their physical and physiological properties in vivo and ex vivo/in vitro.

There is over a 30-year history of the use of dyes in labeling biologically relevant compounds for studying the entire gamut of applications, primarily in vitro, but some in vivo, e.g., optical imaging. Many improvements have been made to the early dyes that were unstable when conjugated to proteins, and also very hydrophobic. Most of these improvements have been made using the sulfonic acid substituent, either on the aryl rings of the dyes, or at the terminus of the aliphatic chains attached, as were many of the successful photographic dyes, primarily addressing issues of water solubility and to some extent the tendency of many dyes to aggregate in solution, and photostability.

In recent years there have been a few instances of the use of dye PEGylation, with some interesting results, but there have not been any reports of a simple, general and broad approach to the PEGylation of dyes using discrete single molecule PEGylation constructs and to do so without also the incorporation of the sulfonic acid substituent. The sulfonic acid substituent, while water soluble, is still not fully compatible with protein and other surfaces, creating issues of non-specific binding. Additionally, the sulfonic acid sticks at the cell surface and inhibits or prevents efficient cell internalization.

The use of discrete PEG linkers have been shown to be extremely effective in modifying the water solubility, as well as to minimize, if not eliminate non-specific binding of otherwise hydrophobic molecules. They are also known and have been shown to not inhibit, but rather facilitate the cell internalization of compounds. They have also shown a strong ability to control or prevent aggregation, as well as have been shown with strongly aggregating proteins like collagen.

Hence providing access a large range of known dye molecules with known and useful photophysical properties for the broad range of the in vivo and in vitro applications for which they are knows, but also ones that are highly water soluble, having little or no non-specific binding, whose cell internalization can be controlled, as well as show low aggregating properties, would be a considerable and valuable advance in the area of diagnostics, including intraoperative optical imaging and theranostics, and even therapy, in areas like photodynamic therapy.

### BRIEF SUMMARY

Generally disclosed are discrete PEGylated dyes, that is, dyes, generally ones that are fluorescent, but could also include chemi- or electrochemiluminescent and related dye or dye precursors, that have discrete PEG constructs chemically attached in various configurations on the dye, and in the entire range of constructs, discrete PEG compounds (polyethylene glycol oligomers that are made synthetically according to methods disclosed in U.S. Pat. Nos. 7,888,536, 8,637,711, and US Pub. No. 2013/0052130, the disclosures of which are expressly incorporated herein by reference). The dyes are modified using arrange of methods to control or optimize the properties of water solubility, non-specific binding (in vitro), biodistribution (in vivo), cell internalization (non-cell or cell based assays in vitro, and in vivo diagnostics and therapy), as well as aggregation. There may be cases, like labeling antibodies for detection purposes, having a dye with only a specifically applied discrete PEG length, e.g., x=12, as the spacer may be a special use case.

Disclosed are discrete PEGs on the cyanine dye backbones, but the dyes are not limited to these, as there is an entire industry of dyes to which this disclosure can be applied. Cyanine dyes are used to demonstrate a representative range of the various constructs for putting on different numbers and different sizes and shapes of discrete PEG constructs, linear and branched, either reactable (selective) or non-reactable, containing inert terminal grouping, or charged, or even other hydrophobic or hydrophilic groups, where these properties can be designed for a particular application, whether it be targeted (preferred) or systemic. The disclosure demonstrates the ability of the discrete PEG to control the efficacy of the dye in an application and create a control group of dyes in order to potentially give some predictability to the design of dyes more generally in biologic applications. The disclosed PEG modified dyes may be represented as follows:

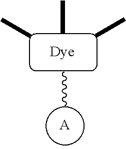

The solid lines as indicated by [symbol] are linear discrete PEG containing constructs which are not terminated by the typical sulfonate or highly charged conjugate bases which are used in most dyes to enhance the water solubility. The solid line will generally be capped with a neutral group, but not limited to a methyl or methoxy group.

The wavy line is a linear discrete PEG containing construct [symbol] , where the terminal A is a reactive or reactable group that is used to attach the said dye to a biological construct that is specifically targeting, and as called presently a preferential locator. The solid line is most often preferable attached to a group on the dye, like a sulfonate, that has been designed in many systems to enhance the stability of the dye and also determine its photophysical properties. These dye chemistries are preferred, but are not limited to the application of this disclosure.

When the solid line, [symbol] , contains a discrete PEG, the range of ethylene oxide units is from about 2 to 64, preferable between about 3 to 24 units.

Optionally, the solid line, [symbol] , in specific cases can be a simple alkyl or similar capping group, for example on a sulfonate, with the purpose of neutralizing the negative charge in the application. The linear discrete PEG of the wavy line, [symbol] , is designed to give the overall construct the water solubility necessary for the application, and can contain non-discrete PEG components, but preferably contains a linear discrete PEG chain ranging from about 2 to 64, or more preferably from about 4 to 24.

The dye can be chosen from those used generally in the in vivo and in vitro applications well known in the art, and referenced in some detail below. Many dyes in these classes have not been used in these in vivo and in vitro biological applications due to physical property limitations, which can be rectified by the incorporation of the proper design of solid and wavy lines disclosed presently.

Shown below are the general classes of the cyanine dyes as a specific application disclosed currently by example.

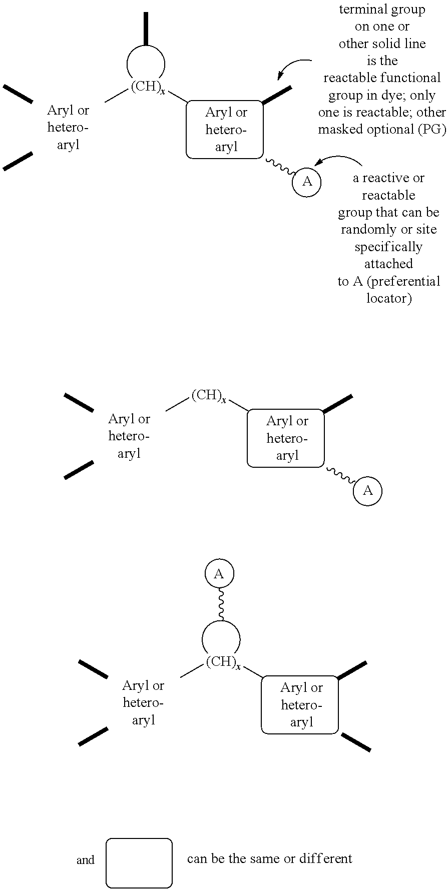

### BRIEF DESCRIPTION OF THE DRAWINGS

For a fuller understanding of the nature and advantages of the present method and process, reference should be had to the following detailed description taken in connection with the accompanying drawings, in which:

FIG. 1 graphically displays the competition ELISA results reported in Example 13;

FIG. 2 graphically displays the fluorescence results reported in Example 14;

FIG. 3 graphically displays additional fluorescence results reported in Example 14; and

FIG. 4 graphically displays cell internalization results reported in Example 14.

The drawings will be described in greater detail below.

### DETAILED DESCRIPTION

### Definitions

### General

The following definitions of terms as used herein are listed below:

Unless defined otherwise, all technical and scientific terms used herein generally have the same meaning as commonly understood by one of ordinary skill in the art to which this disclosure belongs. Generally, the nomenclature used herein and the laboratory procedures in cell culture, protein engineering, molecular genetics, organic chemistry and nucleic acid chemistry, and hybridization described below are those well-known and commonly employed in the art. Standard techniques are used for nucleic acid and peptide synthesis. Generally, enzymatic reactions, protein and related modification and crosslinking chemistry and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art. Standard techniques, or modifications thereof, are used for chemical syntheses and chemical analyses.

“Substantially pure”—This purity is like that of traditional chemical synthesis where the components, which create the various discrete polyethylene glycol (discrete PEG) constructs, are each single compounds. The branched discrete PEG constructs are built from combinations of the individually pure components, [symbol] and [symbol] , in a like manner. The [symbol] and [symbol] are primarily composed of a discrete PEG and derivative made via the processes developed in U.S. Pat. Nos. 7,888,536 and 8,637,711. Additional purification to remove non-discrete PEG impurities can be carried out using conventional purification methodologies where necessary and optimized, especially recrystallization, but also special extractive processing and chromatography, as well. Thus, the disclosed discrete PEG compounds and constructs typically are synthesized in a purity of greater than 60% for those with more complex molecular architecture and often greater than 80% or 90% or above for those with less complicated molecular architecture, especially those that are linear with a side chain G. Methods can generally be developed to make the various disclosed discrete PEG constructs of purities exceeding 97% or 98% and approaching 100%. Even 60% purity is exceedingly higher than the simplest linear monodisperse mixture, where “purity” of the average component is much less than a few % in the best case (PDI=1.01), which are still extremely polydisperse by nature of the polymerization processes by which they are made.

“Wavy line”, “ [symbol] ”. The wavy line, [symbol] , is a linear chain containing a discrete polyethylene glycol (discrete PEG) residue optionally substituted with N, S, Si, Se, or P, and optionally having branching side chains. Such wavy line may contain aryl groups, alkyl groups, amino acids, and the like. The end components of [symbol] have independently chemically reactable or reactive moieties at each end. These are incorporated such that each end can be reacted independently during its incorporation to any discrete PEG construct or intermediates in the process of building the same. When the ends of the wavy line are chemically reactive groups, they can be reactive on their own, or can be masked groups, e.g., an azide as an amine, or protected reactable groups that must be converted to chemically reactive groups. The chemical construction of these compositions can have multiple wavy lines, the same or different. When they are different, the end groups, “A” must not react at the same time, and can be biorthogonal, or other combinations of masked or protected reactable groups known in the art. (Ref.: E. M. Sletten and C. R. Bertozzi, “Biorthogonal Chemistry: Fishing for Selectivity in a Sea of Functionality,” Angew. Chem. Int. Ed., 48, 6974-6998(2009); G. Hermanson, Bioconjugate Techniques, 3rd Edition, Academic Press, 2013; T. Greene and P. Wutz, Greene's Protective Groups in Organic Synthesis, 4^{th} ed., Wiley, 2007.) The use is the same as that disclosed in our U.S. Pat. Nos. 7,888,536 and 8,637,711. Some of the more preferred options are shown in Tables 1 and 2 of U.S. Publ. No. 2013/0052130. The chemically reactable or chemically reactive moieties as end groups on the wavy line also can be converted to biologically active groups. Generally this will be a final step or series of steps in the building of the compositions in this disclosure.

Furthermore, the wavy line [symbol] , which in the art also is termed a linker or spacer or spacer arm, means a chemical moiety comprising a covalent bond or a chain of atoms that covalently attaches a “preferential locator”, like an antibody, or to a diagnostic or therapeutic group, like a drug moiety, or with many dyes, various peptides that are preferential locators. Exemplary linker abbreviations include: MC=6-maleimidocaproyl, MPS=maleimidopropanoyl, val-cit=valine-citrulline, dipeptide site in protease-cleavable linker, ala-phe=alanine-phenylalanine, dipeptide site in protease-cleavable linker, PAB=p-aminobenzyloxycarbonyl, SPP=N-Succinimidyl 4-(2-pyridylthio)pentanoate, SMCC=N-Succinimidyl 4-(Nmaleimidomethyl)cyclohexane-I carboxylate, SIAB=NSuccinimidyl(4-iodo-acetyl)aminobenzoate, and these and others known in the art can be and preferred to be used in the disclose composition containing a linear discrete PEG, as well as those containing discrete PEG constructs described and defined below.

The wavy line [symbol] also is defined such that it contributes important properties to be incorporated into or as part of the composition, as part of controlling and including the length and size of the discrete PEG. These also have practical considerations as they variably control the accessibility for reaction and also the dynamics and size on the final construct, as well as other design functions desirable to the application, e.g., cleavable/releasable, multifunctional. And the optimal lengths of the wavy line are preferred in this disclosure, where for discrete PEGₓ, x is preferred from 2 to 72, more preferred from 8-24. The inherent properties of the discrete PEG as a type of PEG are known in the art.

The wavy line is defined to optionally incorporate a bond or chemical construct known in the art that will result in a cleavable bond or construct. Also see Tables 1 and 2 of U.S. Publ. No. 2013/0052130 for the preferred chemistries to use in this disclosure as part of the definition for the wavy line, [symbol] .

“Solid line,” [symbol] ” solid lines, [symbol] , are discrete PEG-containing chains that have between about 2 and 64 ethylene oxide residues and have a terminal moiety that is not an ethylene oxide. Optionally containing non-discrete PEGs, but a chain having only discrete PEGs is preferred. The terminal group generally will be a methyl group or methoxy group, or a charged group. The composition of the end groups on the solid line can be different. Both ends, independently, also could be chemically reactable group(s) or chemically reactive moiety(s), such that they can be incorporated into a branched, linear or multifunctional composition during a synthetic process, or are as defined above. The solid line can contain aryl, alkyl, etc. groups, but it is preferred that it be a “simple” linear discrete PEG. On occasion, the solid line could incorporate a wavy line or be incorporated into a wavy line. In this disclosure, it is of interest to control the nature of the charge balance, which has a strong impact on properties, such as, for example, cell internalization as well as general non-specific binding, and specifically for cyanine dyes, which when substituted are generally positively charged. However, having the option of putting a negatively or a positively charged group as an end group is part of the definition.

“A” can be a “biologically active group” or a “chemically reactive moiety” or a “chemically reactable moiety.”

“A” as a “Chemically reactive moiety”—a “chemically reactive moiety” is one that will react as it is presented to and allowed to react in the chemical process. This is to be distinguished from a “chemically reactable group” can be used interchangeably, but is a chemical reactive group that is masked, like an azide, reducible to an amine, or a protected “chemically reactive group.”

As used herein, or A—chemically reactive moiety—when two chemically reactive moieties are present in a construct, they are optimally designed to have complimentary reactivity. Hence the A's as “chemically reactive moieties” are a pair of reactive chemical moieties that will by the nature of atoms (well known in the art) react with one another, and designed to only react with each other under the predetermined process conditions in building the branched discrete PEG construct. They are selected from various chemistries known in the art in such a way to give [symbol] the desired chemical, physical or steric properties desired for a particular application as it is built into various discrete PEG constructs architectures. Including and optionally giving the ends or a position in [symbol] the propensity to now be a releasable. Some preferred options are listed, but not limited to, Tables 1 and 2.

When the wavy line is being incorporated initially to a branched core and both ends are “A”, the same is true as the intermolecular reactability above.

Other A's include other sulfhydryl/thiol specific like iodo(halo)acetamides, vinyl sulfone, ETAC (that can react to two thiols, that can be the same or two different in a bispecific application, or bridge a disulfide; bismaleimide or even bis-alphahalo configurations can serve the same and broader function); aminooxy derivatives to react with carbonyls like ketones and aldehydes; acetylides that can react with azides via a copper catalyzed or copper free click reactions, where in the latter case, strained cyclooctynes are most useful, like the BCN or DBCO derivatives. Additionally, tetrazine derivatives and various alkenes, e.g., trans-cyclooctene derivatives are optionally included.

“Chemically reactive moiety” also is a reactive functional group, and as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989). The reactive functional groups may be protected or unprotected (see, for example, Greene's Protective Groups in Organic Synthesis, Peter G. M. Wuts and Theodora W. Greene, John Wiley and Sons, 2007.

The term “Chemically reactable group” as “A” and as used herein is a masked or protected “chemically reactive group” and used such that where more than one “A” is in a method for making the various discrete PEG constructs, these do not interfere in the successful outcome of the syntheses. These options for having “chemically reactable groups” in the presence of “chemically reactive groups” are well known in the art. Many of these are shown in Tables 1 and 2 of U.S. Pub. No. US 2013/0052130.

Most of the chemically reactive moieties most preferred in this disclosure can be found in application in the representative references by Hermanson and Bertozzi, but not limited to these, and many are well known to those skilled in the art. (Ref.: Bioconjugate Techniques, Greg T. Hermanson, 3^{rd} ed., Elsevier, 2013; ISBN 978-0-12-382239-0; “Biorthogonal Chemistry: Fishing for Selectivity in a Sea of Functionality,” Ellen M. Sletten and Carolyn R. Bertozzi, Angewandte Chemie Int. Ed., 2009, 48, 6974-6998.)

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality.

Suitable protecting groups include acetyl, benzyl, benzoyl, tetrahydropyranyl, and trialkylsilyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitro ethyl and the like. For a general description d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process (Ref.: Greene's Protective Groups in Organic Synthesis, Peter G. M. Wuts and Theodora W. Greene, John Wiley and Sons, 2007.

“A” as a “Biologically active group”—This is a biologically active group that is either able to target (preferential locator) a particular compound that is matched to A with a specific non-covalent affinity, e.g., or one that can interact with a target in specific and complementary ways, e.g., enzyme inhibitor peptide (A) to an enzyme released at a disease sight. Any of these biologically active groups inhibitor can be delivered with a radiolabel or a toxic drug that would kill the target, or can deliver a detectable probe as a diagnostic agent, or both.

“A” as a biologically active group is introduced into the discrete PEG constructs by the many chemistries known in the art, e.g., references: E. M. Sletten and C. R. Bertozzi, “Biorthogonal Chemistry: Fishing for Selectivity in a Sea of Functionality,” Angew. Chem. Int. Ed., 48, 6974-6998(2009); G. Hermanson, Bioconjugate Techniques, 3rd Edition, Academic Press, 2013. In addition the option for incorporating a cleavable chemistry into the linkage formed also is a preferred option. This could include but not limited to a cleavable peptide, a disulfide, or a hydrazone.

As used herein, “A” can be a targeting agent, or carrier with targeting agent (e.g., a nanoparticle that has the targeting agents attached to the particle with various linear and branched discrete PEG constructs), the targeting agent matched to a particular target. A can be, e.g., a MMP (matrix metalloprotease) inhibitor substrate, an RGD peptide, antibody, antibody fragment, engineered scaffold, liposome, a PLGA, silica or a metal nanoparticle, such as gold or silver, all well known in the art or targeting for diagnostics and therapeutics.

When there is more than one “A” as a “biologically active group”, the term used is a multivalent group. The “A” independently can be the same or different depending on the intent and need of the particular application of “A”. E.g., Two different “A's” give a bispecific interaction, or where “A” is the same, a single interaction can be enhanced, but in both cases there can be a very large advantage over having just one “A” and the design of the [symbol] can control that synergy of having more than one “A.”

The term, Terminal moiety, as used herein in this disclosure, is defined in terms of the group at the end of the solid line, [symbol] , in a branched or linear discrete PEG construct. Preferred groups are the methyl or methoxy, and the carboxyl/carboxylate. In certain cases, the terminal group can be a positively charged group, like guanidine, amine and the like, including short peptides, or an amine or a quaternary ammonium moiety. In the case of multiple [symbol] 's, various combinations can be used also in order to control the charge balance as well as the presently stated properties. These groups may control cell penetration either positively or to prevent it, as well as the orientation and geometry of the variously disclosed discrete PEG constructs. Having multiple charged terminal groups, especially the carboxyl, is preferred in controlling the biodistribution, to unexpectedly increasing the apparent size of a branched discrete PEG construct or multiple linear discrete PEG constructs in close proximity, and thereby give “small” constructs that will not go out the kidney and stay out of other organs, as well and thereby control much of the biodistribution of a branched or multiple linear discrete PEG construct having “A” with a biologically active group attached and thereby direct the biologically active group to a preferred location very specifically without diversion while carrying a diagnostic or therapeutic or both groups, and also control the PK of the final branched discrete PEG construct.

Charged group: A charged group or groups are functional groups that have a net positive or negative charge. The presence and nature of the charge is generally dictated by the pH of the environment in which the group is found. E.g., at physiological pH of just above 7 the amine group is positive and the carboxylate is negative, as are the phosphate and sulfonate groups. Other positively charged groups may include guanidine or specific quarteranized amines. The preferred function is the same as for the terminal group, where the preferred terminal group is negatively charged, more preferred the carboxyl group, but optionally having a positive charge. Some discrete PEG constructs may be designed having both negative and positive charges in them by design.

As used herein, “G” means a protected or masked reactive chemical moiety; “reactive chemical moiety”=group of atoms that will react with another group of atoms to form the desired chemical bond or bonds based on the electronic and/or steric nature of the reacting group of atoms. “G” has the same options at “A” (chemically reactive group above), but have defined it separately to distinguish it as reactive functionality coming off of template AC's wavy lines. (Refs.: a) March's Advanced Organic Chemistry: Reactions, Mechanism and Structure, Michael B. Smith and Jerry March, John Wiley & Sons, 2001; b) Greene's Protective Groups in Organic Synthesis, Peter G. M. Wuts and Theodora Greene, 4^{th} ed., John Wiley & Sons, 2007.) “G” is convertible to a “G” that can be a “DG”, diagnostic group, or a “TG”, a therapeutic group, but is not limited to groups with just these functionality and applicability.

### Diagnostic Group

The term “diagnostic group”, abbreviated “DG,” which is used interchangeably with “detectable label” is intended to mean a moiety having a detectable physical, chemical, or magnetic property. This includes such labels as biotin and its derivatives, which are matched with the entire range of streptavidin conjugates, dyes, fluorescent and chromogenic, radioisotopes as labels, including chelating groups such as DOTA and NOTA derivative. In all of these cases the use of the linear discrete PEG in the attachment chemistry is preferred. (Ref.: a. D. Scott Wilbur, “Chemical and Radiochemical Considerations in Radiolabeling with alpha-Emitting Radionuclides,” Current Radiopharmaceuticals, 4, 214-247(2011); M. Famulok, et al., “Functional Aptamers and Aptazymes in Biotechnology, Diagnostics, and Therapy,” Chem. Rev., 107(9), 3715-3743(2007); S. S. Kelkar and T. M. Reineke, “Theranostics: Combining Imaging and Therapy,” Bioconjugate Chemistry, 22, 1879-1903); “Molecular Probes Handbook, A Guide to Fluorescent Probes and Labeling Technologies,” 11^{th} Edition, Iain Johnson and M. Spence, Ed., ISBN-10: 0982927916.

### TG (Therapeutic Group)

The term “therapeutic (group)” abbreviated “TG,” is intended to mean a compound that, when present in a therapeutically effective amount, produces a desired therapeutic effect on a mammal. For treating carcinomas, it is desirable that the therapeutic agent also be capable of entering the target cell. A therapeutic group can be from among the cytotoxins. Herein, the term “cytotoxin” is intended to mean a therapeutic agent having the desired effect of being cytotoxic to cancer cells. Cytotoxic means that the agent arrests the growth of or kills the cells. Exemplary cytotoxins include, by way of example and not limitation, combretastatins, duocarmycins, the CC-1065 anti-tumor antibiotics, anthracyclines, and related compounds. Other cytotoxins include mycotoxins, ricin and its analogues, calicheamycins, doxirubicin and maytansinoids. A good recent review reference on natural products and their potential impact on new anti-cancer drugs is referenced here. (“Impact of Natural Products on Developing New Anti-Cancer Agents,” David J. Newman, et al., Chemical Reviews, 2009, 109, 3012-3043.

As used herein, the term “therapeutic group” is any compound that is a “drug”, “anticancer agent”, “chemotherapeutic agent”, “antineoplastic”, and “antitumor agent” are used interchangeably and refer to agent(s) (unless further qualified) that have the property of inhibiting or reducing aberrant cell growth, e.g., a cancer. The foregoing terms also are intended to include cytotoxic, cytocidal, or cytostatic agents. The term “agent” includes small molecules, macromolecules (e.g., peptides, proteins, antibodies, or antibody fragments), and nucleic acids (e.g., gene therapy constructs), recombinant viruses, nucleic acid fragments (including, e.g., synthetic nucleic acid fragments). (Ref.: M. Famulok, “Functional Aptamers and Atazymes in Biotechnology, Diagnostics, and Therapy,” Chem. Rev., 107(9), 3715 (2007).

Therapeutic groups also can be radionuclides (Refs.: D. Scott Wilbur, “Chemical and Radiochemical Considerations in Radiolabeling with -Emitting Radionuclides,” Current Radiopharmaceuticals, 4, 214-247(2011); Monoclonal antibody and peptide-targeted radiotherapy of cancer, R. M. Reilly, ed., J. Wiley and Sons, 2010, ISBN 978-0-470-24372-5; c. Targeted Radionuclide Therapy, Tod W. Speer, ed., Lippincott, 2011, ISBN 978-0-7817-9693-4.)

Nanoparticle

As used herein, the term “nanoparticles” refers to particles of about 0.1 nm to about 1 μm, 1 nm to about 1 μm, about 10 nm to about 1 μm, about 50 nm to about 1 μm, about 100 nm to about 1 μm, about 250-900 nm in size, or, advantageously, about 600-800 nm. The nanoparticles may comprise macromolecules, gene therapy constructs, or chemotherapeutic agents, for example.

As used herein, the term “microparticles” refers to particles of about 0.1 μm to about 100 μm, about 0.5 μm to about 50 μm, 0.5 μm to about 20 μm in size, advantageously, particles of about 1 μm to about 10 μm in size, about 5 μm in size, or mixtures thereof. The microparticles may comprise macromolecules, gene therapy constructs, or chemotherapeutic agents, for example.

The term “cleavable group” is intended to mean a moiety that can be unstable in vivo. Preferably the “cleavable group” allows for activation of the marker or therapeutic agent by cleaving the marker or agent from the rest of the conjugate. Operatively defined, the linker is preferably cleaved in vivo by the biological environment. The cleavage may come from any process without limitation, e.g., enzymatic, reductive, pH, etc. Preferably, the cleavable group is selected so that activation occurs at the desired site of action, which can be a site in or near the target cells (e.g., carcinoma cells) or tissues such as at the site of therapeutic action or marker activity. Such cleavage may be enzymatic and exemplary enzymatically cleavable groups include natural amino acids or peptide sequences that end with a natural amino acid, and are attached at their carboxyl terminus to the linker. While the degree of cleavage rate enhancement is not critical to the disclosure, preferred examples of cleavable linkers are those in which at least about 10% of the cleavable groups are cleaved in the blood stream within 24 hours of administration, most preferably at least about 35%. Included in this term is the option of having a “self immolative spacer”. The term “self-immolative spacer” refers to a bifunctional chemical moiety that is capable of covalently linking two chemical moieties into a normally stable tripartite molecule. The self-immolative spacer is capable of spontaneously separating from the second moiety if the bond to the first moiety is cleaved. Listed are references representing the range of cleavable chemistries potentially applicable in this disclosure, which can be utilized with the benefit by incorporation into the wavy or solid lines, especially containing discrete PEGs, as part of a branched core or the attachment core.
a. “Releasable PEGylation of proteins with customized linkers,” David Filpula and Hong Zhao, Advances in Drug Delivery Reviews, 2008, 60, 29-49.
b. “A Mild Chemically Cleavable Linker System for Functional Proteomic Applications,” Steven H. L. Verhelst, Marko Fonovic', and Matthew Bogyo, Angew. Chem. Int. Ed. 2007, 46, 1-4.
c. “Enzyme-Catalyzed Activation of Anticancer Prodrugs,” MARTIJN ROOSEBOOM, JAN N. M. COMMANDEUR, AND NICO P. E. VERMEULEN, Pharmacol Rev 56:53-102, 2004.
d. “Elongated Multiple Electronic Cascade and Cyclization Spacer Systems in Activatible Anticancer Prodrugs for Enhanced Drug Release,” Hans W. Scheeren, et al., J. Org. Chem. 2001, 66, 8815-8830.
e. “Controlled Release of Proteins from Their Poly(Ethylene Glycol) Conjugates: Drug Delivery Systems Employing 1,6-Elimination,” Richard B. Greenwald, et al., Bioconjugate Chem. 2003, 14, 395-403.

The term “pro drug” and the term “cleavable moiety” often can be used herein interchangeably. Both refer to a compound that is relatively innocuous to cells while still in the conjugated form, but which is selectively degraded to a pharmacologically active form by conditions, e.g., enzymes, located within or in the proximity of target cells. (Refs.: P. J. Sinko, et al., “Recent Trends in Targeted Anticancer Prodrug and Conjugate Design,” Curr. Med. Chem., 15(18), 1802-1826(2008); S. S. Banerjee, et al., Poly(ethylene glycol)-Prodrug Conjugates: Concept, Design, and Applications,” J. of Drug Delivery, Article ID 103973 (2012); J. Rautio, et al., “Prodrugs: design and clinical applications,” Nature Review, Drug Discovery, 7, 255-270 (2008).)

Preferential locator often can be used largely interchangeably with ligand or “targeting group” and can be either a “diagnostic group” or a “therapeutic group” or the like. Broadly, preferential locators are molecularly targeted agent defined as drugs that target growth factor receptors and signal transduction pathways. NPOA molecule is used for targeting molecular entities, cells, tissues or organs in a biological system. With respect to neoplastic tissue (cancer cells), a “preferential locator” (or “locator”) specifically binds a marker produced by or associated with, for example, neoplastic tissue, antibodies and somatostatin congeners being representative such locators. Broader, however, a “locator” includes a substance that preferentially concentrates at the tumor sites by binding with a marker (the cancer cell or a product of the cancer cell, for example) produced by or associated with neoplastic tissue or neoplasms. Appropriate locators today primarily include antibodies (whole and monoclonal), antibody fragments, chimeric versions of whole antibodies and antibody fragments, and humanized versions thereof. It will be appreciated, however, that single chain antibodies (SCAs, such as disclosed in U.S. Pat. No. 4,946,778, incorporated herein by reference) and like substances have been developed and may similarly prove efficacious. For example, genetic engineering has been used to generate a variety of modified antibody molecules with distinctive properties. These include various antibody fragments and various antibody formats. An antibody fragment is intended to mean any portion of a complete antibody molecule. These include terminal deletions and protease digestion-derived molecules, as well as immunoglobulin molecules with internal deletions, such as deletions in the IgG constant region that alter Fc mediated antibody effector functions. Thus, an IgG heavy chain with a deletion of the Fc CH2 domain is an example of an antibody fragment. It is also useful to engineer antibody molecules to provide various antibody formats. In addition to single chain antibodies, useful antibody formats include divalent antibodies, tetrabodies, triabodies, diabodies, minibodies, camelid derived antibodies, shark derived antibodies, and other antibody formats. Aptomers form yet a further class of preferential locators. All of these antibody-derived molecules are example of preferential locators.

Various suitable antibodies (including fragments, single chains, domain deletions, humanized, etc.) include, for example, B72.3, CC49, V59, and 3E8 (see U.S. Pat. No. 8,119,132), all directed against adenocarcinomas.

In addition to antibodies, biochemistry and genetic engineering have been used to produce protein molecules that mimic the function of antibodies. Avimers are an example of such molecules. See, generally, Jeong, et al., “Avimers hold their own”, Nature Biotechnology Vol. 23 No. 12 (December 2005). Avimers are useful because they have low immunogenicity in vivo and can be engineered to preferentially locate to a wide range of target molecules such as cell specific cell surface molecules. Although such substances may not be subsumed within the traditional definition of “antibody”, avimer molecules that selectively concentrate at the sites of neoplastic tissue are intended to be included within the definition of preferential locator. Thus, the terms “locator” was chosen, to include present-day antibodies and equivalents thereof, such as avimers, as well as other engineered proteins and substances, either already demonstrated or yet to be discovered, which mimic the specific binding properties of antibodies in the inventive method disclosed therein. (Refs.: “Engineered protein scaffolds as next-generation antibody therapeutics,” Michaela Gebauer and Arne Skerra, Current Opinion in Chemical Biology, 2009, 13, 245-255; “Adnectins: engineered target-binding protein therapeutics,” D Lipovsek, Protein Engineering, Design & Selection, 2010, 1-7.)

For other disease types or states, other compounds will serve as preferential locators.

The term “preferential locator” also can include terms like “targeting group” and “targeting agent” and are intended to mean a moiety that is (1) able to direct the entity to which it is attached (e.g., therapeutic agent or marker) to a target cell, for example to a specific type of tumor cell or (2) is preferentially activated at a target tissue, for example a tumor. The targeting group or targeting agent can be a small molecule, which is intended to include both non-peptides and peptides. The targeting group also can be a macromolecule, which includes saccharides, lectins, receptors, ligands for receptors, proteins such as BSA, antibodies, and so forth. (Refs.: a) “Peptides and Peptide Hormones for Molecular Imaging and Disease Diagnosis,” Xiaoyuan Chen, et al., Chemical Reviews, 2010, 110, 3087-3111; b) “Integrin Targeted Therapeutics,” N. Neamati, et al., Theranostics, 2011, 1, 154-188; c) “Integrin Targeting for Tumor Optical Imaging,” Yunpeng Ye, et al., Theranostics, 2011, 1, 102-126.)

The term “marker” is intended to mean a compound useful in the characterization of tumors or other medical condition, and is therefore a target for the “preferential locator”. E.g., in the cases of the, diagnosis, progression of a tumor, and assay of the factors secreted by tumor cells. Markers are considered a subset of “diagnostic agents.” (Ref.: “Antibody-Drug Conjugate Targets,” B. A. Teicher, Current Cancer Drug Targets, 2009, 9, 982-1004.) Marker is one target, a major target of a preferential locator. The term “ligand” means any molecule that specifically binds or reactively associates or complexes with a receptor, substrate, antigenic determinant, or other binding site on a target cell or tissue. Examples of ligands include antibodies and fragments thereof (e.g., a monoclonal antibody or fragment thereof), enzymes (e.g., fibrinolytic enzymes), biologic response modifiers (e.g., interleukins, interferons, erythropoietin, or colony stimulating factors), peptide hormones, and antigen-binding fragments thereof. (Ref.: U.S. Pat. No. 7,553,816 B2).

The terms “polypeptide,” “peptide” and “protein” are used interchangeably herein to refer to a defined polymer of amino acid residues, optionally incorporating a discrete PEG spacer or side chain. The terms apply to defined amino acid polymers in which one or more amino acid residue is an artificial chemical mimetic of a corresponding naturally occurring amino acid, as well as to naturally occurring defined amino acid polymers and non-naturally occurring amino acid sequences.

The term “amino acid” refers to naturally occurring and synthetic amino acids, either of L- or D-stereochemical configurations. (Refs.: U.S. Pat. No. 7,553,816 B2; Chang C. Liu and Peter G. Schultz, “Adding New Chemistries to the Genetic Code,” Annu. Rev. Biochem., 79, 413-444(2010)).

The following references are cited as diagnostic, imaging, and therapeutic examples, which alone or in combination, that can be used as the biology and chemistry base into which or upon which, or can be constructed in multiples in addition to a single unit, the discrete PEG constructs taught in this disclose can be designed to give the unexpected and dramatic improvements that have been shown in some very simple cases. Such references are expressly incorporated into this disclosure by reference.

### REFERENCES

(1) Mujumdar, R. B., Ernst, L. A., Mujumdar, S. R., Lewis, C. J., and Waggoner, A. S. (1993) Cyanine Dye Labeling Reagents: Sulfoindocyanine Succinimidyl Esters. Bioconjugate Chem. 4, 105-111.
(2) Ernst, L. A., Gupta, R. K., Mujumdar, R. B., and Waggoner, A. S. (1989) Cyanine Dye Labeling Reagents for Sulfhydryl groups. Cytometry 10, 3-10.
(3) Mujumdar, R. B., Ernst, L. A., Mujumdar, S. R., and Waggoner, A. S. (1989) Cyanine Dye Labeling Reagents containing Isothiocyanate groups. Cytometry 10, 11-19.
(4) Southwick, P. L., Ernst, L. A., Tauriello, E. W., Stephen, R. P., Mujumdar, R. B., Mujumdar, S. R., Clever, H. A., and Waggoner, A. S. (1990) Cyanine Dye Labeling reagents—Carboxymethylindocyanine Succinimidyl Esters. Cytometry, 11, 418-430.
(5) Hamer, F. M. (1964) The Cyanine Dyes and Related Compounds, Wiley, New York.
(6) Narayanan, N., and Patonay, G. (1995) A New Method for the Synthesis of Heptamine Cyanine Dyes: Synthesis of New Near-Infrared Fluorescent Labels. J. Org. Chem. 60, 2391-2395.
7) Illy, H., and Funderburk, L. (1968). Fisher Indole Synthesis Direction of Cyclization of Isopropylmethyl Ketone Phenyl hydrazone. J. Org. Chem. 33, 4283-4285.
(8) Heseltine, D. W., Jones, J. E., and Lincoln, L. L. (1969), Butadienyl Dyes for Photography, U.S. Pat. No. 3,481,927.
(9) Sturmer, D. M. (1977) Syntheses and Properties of Cyanine and Related dyes. In Special Topics in Heterocyclic Chemistry (W. T. Weissberger and E. C. Taylor, Eds.) pp 441-587, John Wiley & Sons, New York.
(10) Meguellati, K., Koripelly, G., and Ladame, S. (2010) DNAtemplated synthesis of trimethine cyanine dyes: a versatile fluorogenic reaction for sensing G-quadruplex formation. Angew. Chem., Int. Ed. Engl., 49, 2738-2742.
(11) Lee, H., Mason, J. C., and Achilefu, S. (2008) Synthesis and spectral properties of near-infrared aminophenyl-, hydroxyphenyl-, and phenyl-substituted heptamethine cyanines. J. Org. Chem. 73, 723-725.
(12) Lee, H., Akers, W., Bhushan, K., Bloch, S., Sudlow, G., Tang, R., and Achilefu, S. (2011) Near-infrared pH-activatable fluorescent probes for imaging primary and metastatic breast tumors. Bioconjugate Chem. 22, 777-784.
(13) Pauli, J., Vag, T., Haag, R., Spieles, M., Wenzel, M., Kaiser, W. A., Resch-Genger, U., and Hilger, I. (2009) An in vitro characterization study of new near infrared dyes for molecular imaging. Eur. J. Med. Chem. 44, 3496-3503.
(14) Kobayashi, H., and Choyke, P. L. (2011) Target-cancer-cell specific activatable fluorescence imaging probes: rational design and in vivo applications. Acc. Chem. Res. 44, 83-90.
(15) Lee, S., Xie, J., and Chen, X. (2010) Activatable molecular probes for cancer imaging. Curr. Top. Med. Chem. 10, 1135-1144.
(16) Chen, K., and Chen, X. (2010) Design and development of molecular imaging probes. Curr. Top. Med. Chem. 10, 1227-1236.
(17) Gragg, Jamie Loretta, “Synthesis of Near-Infrared Heptamethine Cyanine Dyes” (2010). Chemistry Theses. Paper 28. http://digitalarchive.gsu.edu/chemistry_theses/28 and references therein.

In reference (17) and in addition to Hamer's book reference, is a fairly representative history of cyanine and related dye synthesis. In many of the examples that do not have the structures, we show in the Schemes represented primarily by the indocyanine dyes, the substitutions for sulfonates and alkyl sulfonates, alkyl, alkyl carboxyl, alkylamino and other amino substituted groups can be substituted with the R1, R2, R3 and R4 disclosed for the schemes as various linear discrete PEGs, terminated with various groups, and optionally with sulfonate or amines or substituted amines, or small peptides or other species that can control the properties of the dye construct especially in vivo, especially for controlling biodistribution and cell internalization of the dye or its attachments.
(18) Fernando, Nilmi T., “Novel Near-Infrared Cyanine Dyes for Fluorescence Imaging in Biological Systems” (2011). Chemistry Dissertations. Paper 57 and references therein.

Shown in Schemes 1-4 are examples of the various permutations of the discrete PEG constructs in a set of cyanine dyes

Scheme 1: Shows the options in preparing a range of useful discrete PEG dyes that have absorption properties in the visible and NIR spectral range and useful for many light and imaging sources, e.g., exciting lasers, for a broad range of applications.

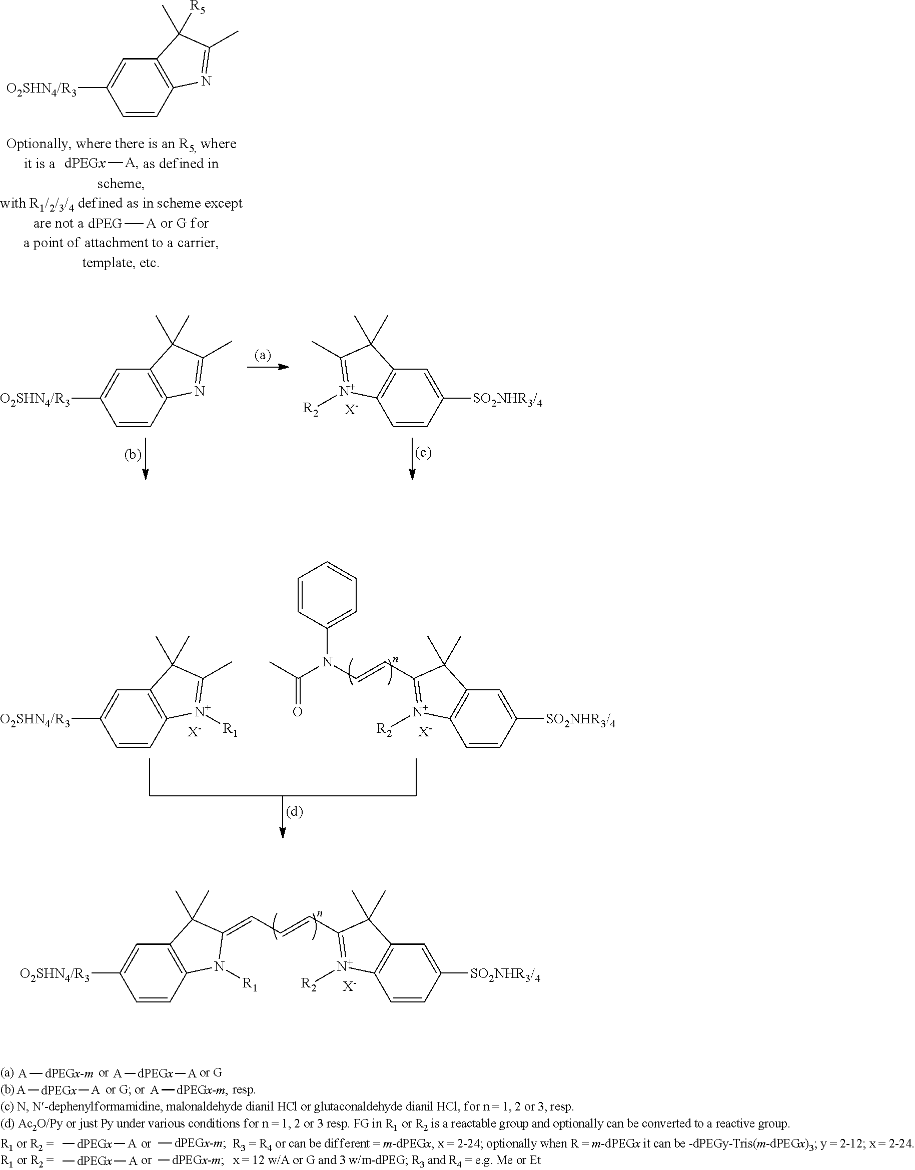

Scheme 2: Is similar to Scheme 1, but shows a set of dyes with a different range of absorption properties useful in the art.

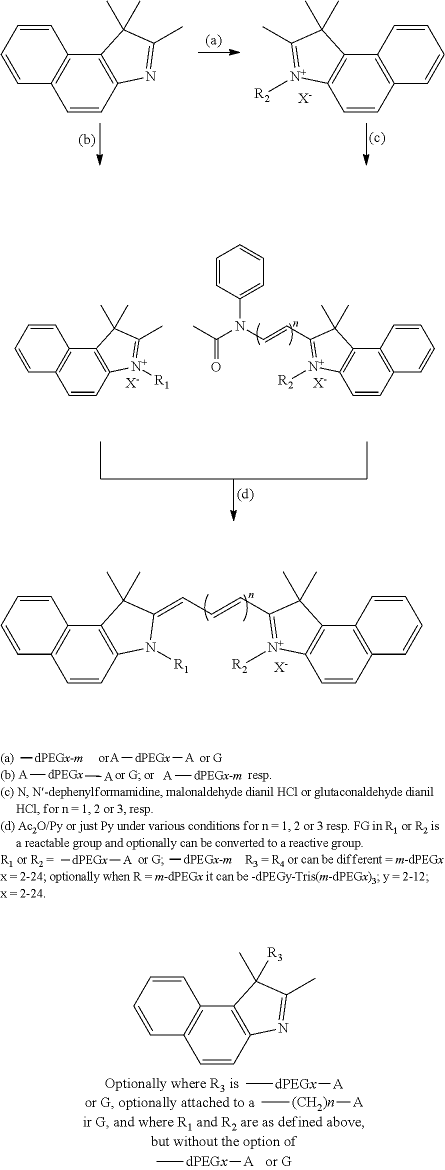

Scheme 3: Showing precursors with aromatic substitution that can be applied as shown in Scheme 2. Examples of the versatility of the discrete PEG construct in controlling the physical properties of dyes.

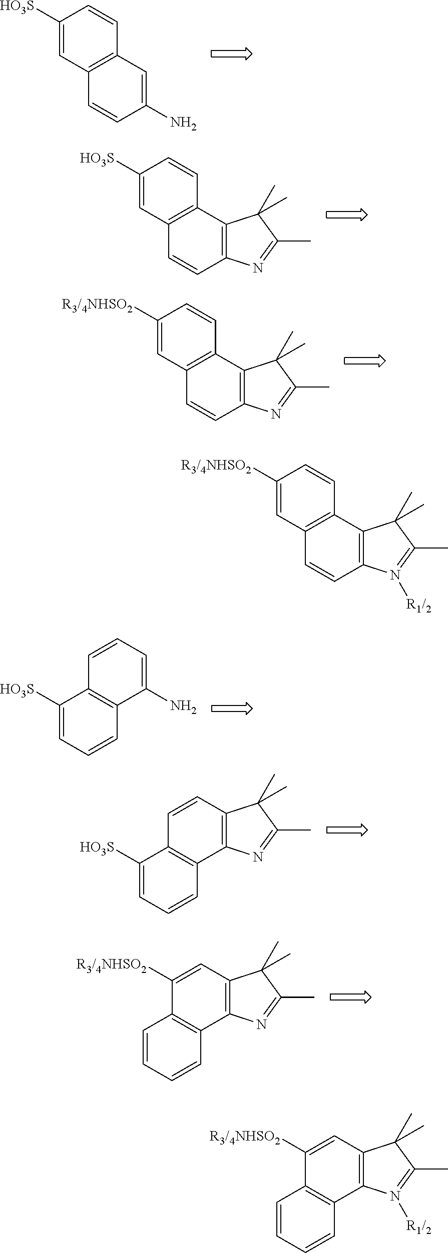

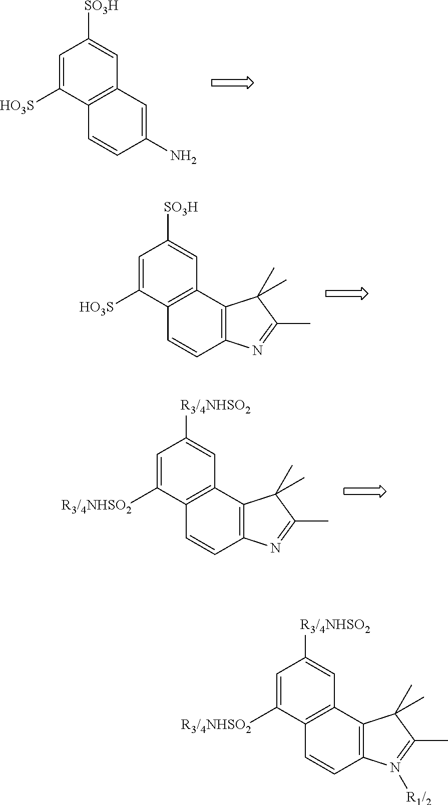

Scheme 4: NIR dyes with the heptamethine grouping between the aromatic ends of the dye have the generally preferred option of putting the reactable group in the middle of the heptamethine grouping, either directly on a methane or as part of a grouping that causes the bridge to be rigid, which enhances the photophysical properties of the dye and where R_{3/4}= [symbol] ; R_{1/2}=—or

[symbol]

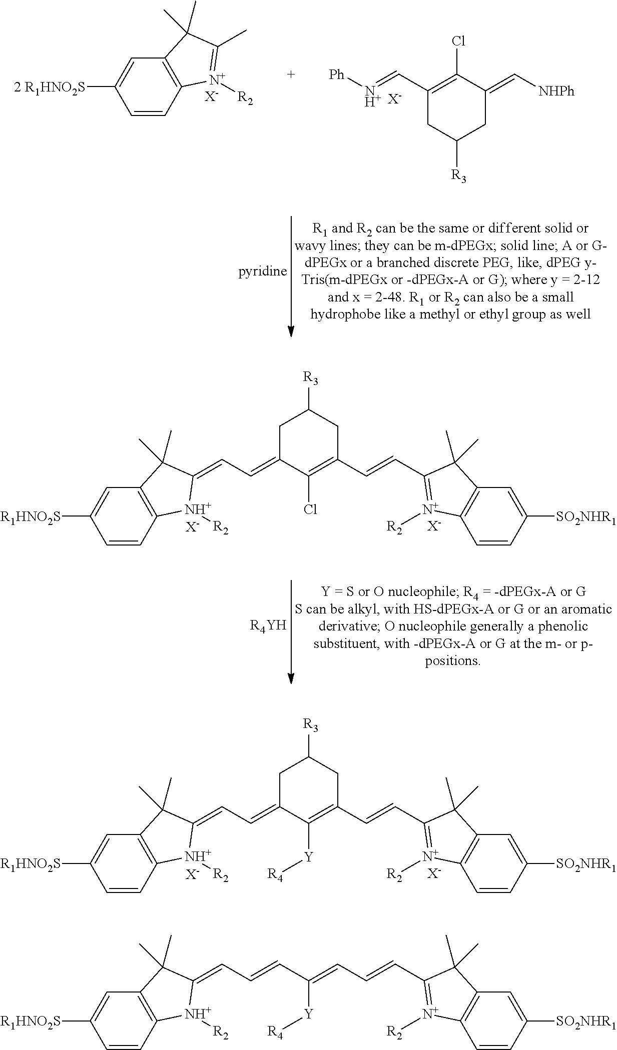

However, while the cyanine dyes serve as a foundation to demonstrate the use of the dPEG in a dye set, and have the most extensive chemical and application history, the use of the dPEG generally without having the sulfonate or related strong conjugate acid salt for modifying the water solubility of the dye, we can propose a more general structure of a dye, that does not contain these moieties on it, thought since they often provide photostability to the dyes, they can be capped with either an alky group of a dPEG construct as defined for the wavy and solid lines. This more general structure is shown below.

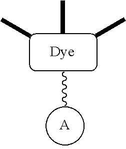

### EXAMPLES

### Example 1

### m-dPEG₃-1,1,2-trimethyl-benzoindolium bromide

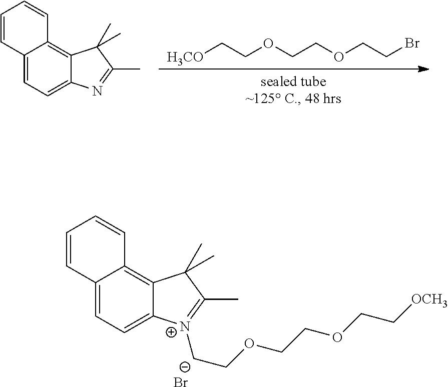 [SMILES: CC1=Nc2ccc3ccccc3c2C1(C)C ; COCCOCCOCCBr ; COCCOCCOCC[N+]1=C(C)C(C)(C)c2c1ccc1ccccc21 ; [Br-]]

A mixture of 7.9 g (37.7 mmol) of 1,1,2-trimethyl-1H-benz[e]indole and m-dPEG₃-Br (12.8 g, 56.4 mmol) in 90 mL of acetonitrile was charged in a 250 mL glass pressure reactor equipped with a magnetic stirrer and heated at 125° C. in an oil bath for 48 hrs. After cooling the reaction to ambient temperature the solvent was removed under reduced pressure to afford 24.5 g of dark viscous oil. The crude was purified by column chromatography on silica gel using gradient elution with dichloromethane-methanol mixture to give 8.8 g (52%) of product as dark glassy green oil.

¹H NMR (400 MHz, CDCl₃, δ): 8.13 (d, 1H, aromatic), 8.05 (d, 1H, aromatic), 8.00 (d, 1H, aromatic), 7.95 (d, 1H, aromatic), 7.68 (t, 1H, aromatic), 7.59 (t, 1H, aromatic), 5.17 (t, 2H, CH₂—N), 4.06 (t, 2H, CH₂O), 3.52-3.28 (m, 8H, CH₂O), 3.21 (s, 3H, CH₃), 3.11 (s, 3H, CH₃), 1.81 (s, 6H, CH₃).

### Example 2

### m-dPEG₃-1,1-dimethyl-N-phenylacetamido-hexa-1,3,5-trienyl-benzoindolium bromide

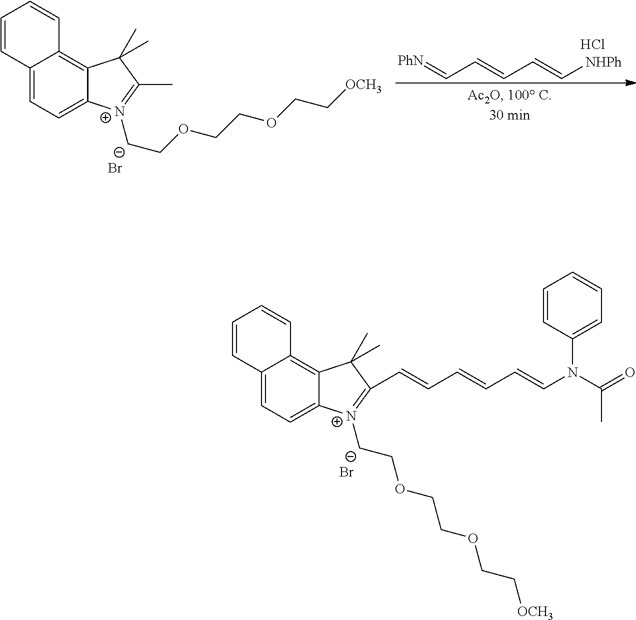

A mixture of the above benzoindolium salt (8.8 g, 20.17 mmol) and glutaconaldehyde dianyl (7.5 g, 26.3 mmol) in 120 mL of acetic anhydride was charged in a 250 mL three-neck round bottom flask equipped with a magnetic stirrer, thermocouple, condenser, nitrogen bleed, and heating mantle. The mixture was heated at 10° C. for 30 min resulting in completion of the reaction, the mixture was transferred into a 0.5 L one-neck round bottom flask, and a majority of acetic anhydride was removed under reduced pressure. The obtained dark residue was co-evaporated with toluene (2×80 mL), and the obtained crude (19.8 g) was purified by column chromatography on silica gel using gradient elution with dichloromethane-methanol mixture to give 10.4 g (81% yield) of product as a dark green amorphous solid.

¹H NMR (400 MHz, CDCl₃, δ): 8.18-8.11 (m, 2H, aromatic, CH═CH), 8.03-7.97 (m, 3H, CH═CH), 7.85 (d, 1H, CH═CH), 7.78 (d, 1H, aromatic), 7.69 (t, 1H, aromatic), 7.60 (t, 1H, aromatic), 7.55-7.48 (m, 3H, aromatic), 7.25 (t, 1H), 7.14-7.08 (m, 2H, aromatic), 6.90 (t, 1H), 5.38 (t, 1H), 5.16 (t, 2H, CH₂—N), 4.08 (t, 2H, CH₂O), 3.56 (m, 2H, CH₂O), 3.41 (m, 2H, CH₂O), 3.34 (m, 2H, CH₂O), 3.24 (m, 2H, CH₂O), 3.18 (s, 3H, CH₃), 1.98 (s, 6H, CH₃), 1.95 (s, 3H, CH₃CO).

### Example 3

### Bis-(m-dPEG₃-1,1-dimethyl-benzoinoliden)-hepta-1,3,5-trienyl-(dPEG₁₂-TBE-1,1-dimethyl-benzoindolium)bromide

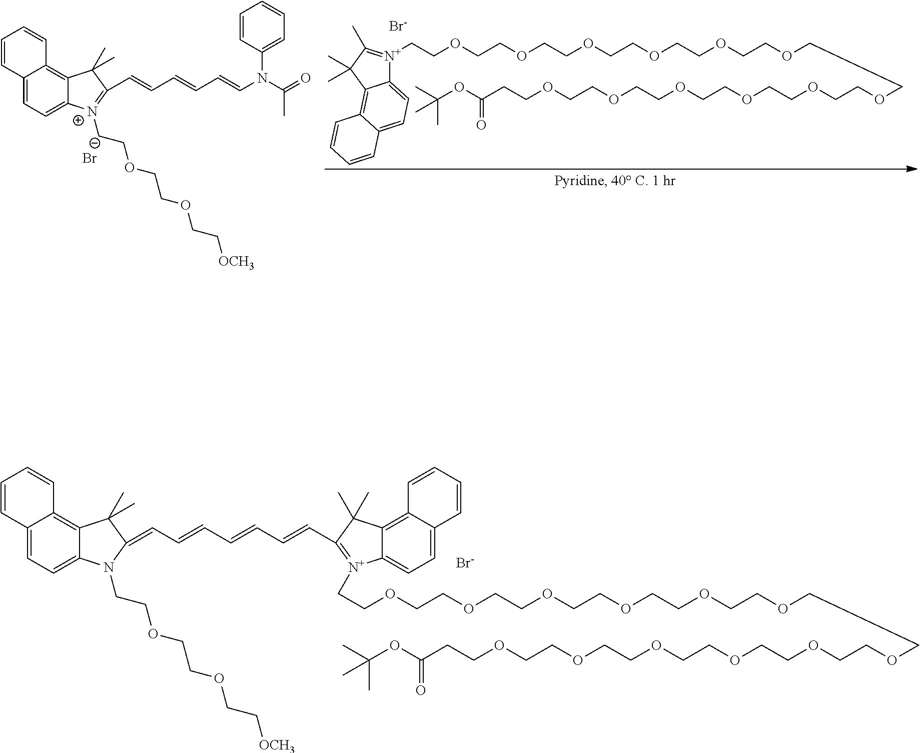

A mixture m-dPEG₃-N-phenylacetamido-hexa1,3,5-trienyl-benzoindolium bromide (2.28 g, 3.60 mmol) and 1,1,2-trimethyl-benzoindolium-dPEG₁₂ bromide (3.39 g, 3.58 mmol) in 45 mL of anhydrous pyridine was charged in a 100 mL three-neck round bottom flask equipped with a magnetic stirrer, thermocouple, condenser, nitrogen bleed, and heating mantle. The mixture was heated at 40° C. for 60 min resulting in completion of the reaction, cooled to ambient temperature, and pyridine was removed under reduced pressure. The residue was diluted with dichloromethane (150 mL) and washed with cold water (2×100 mL). The bottom organic layer was separated, the aqueous phase was extracted with dichloromethane (2×60 mL), and the combined organic extracts were dried over anhydrous sodium sulfate. Drying agent was removed by filtration, and the filtrate was concentrated on rotavap to give 6 g of crude material as green oil. The crude was purified by column chromatography on silica gel using gradient elution with dichloromethane-methanol mixture to give 3.8 g (78% yield) of product as a viscous green foam.

¹H NMR (400 MHz, CDCl₃, δ): 8.11 (d, 2H, aromatic), 7.89 (t, 6H, aromatic/CH═CH), 7.59 (t, 2H, aromatic/CH═CH), 7.51 (d, 2H, aromatic), 7.44 (t, 2H, aromatic), 6.67 (t, 2H), 6.44 (broad s, 2H), 4.47 (broad s, 4H), 3.97 (t, 4H), 3.73-3.45 (m, 58H, CH₂O), 3.39-3.33 (m, 2H), 3.27 (s, 3H, CH₃O), 2.49 (t, 2H, CH₂CO), 1.99 (s, 12H, CH₃), 1.44 (s, 9H, t-Bu).

### Example 4

Bis-(m-dPEG₃-1,1-dimethyl-benzoinoliden)-hepta-1,3,5-trienyl-(dPEG₁₂-acid-1,1-dimethyl-benzoindolium)bromide

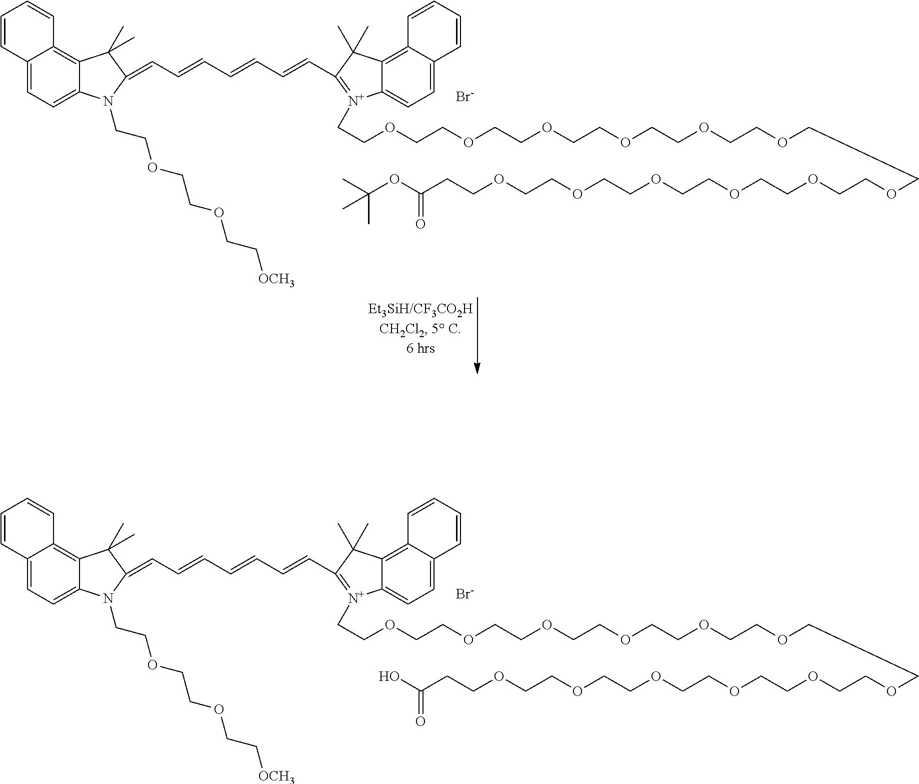 [SMILES: COCCOCCOCCN1\C(=C/C=C/C=C/C=C/C2=[N+](CCOCCOCCOCCOCCOCCOCCOCCOCCOCCOCCOCCOCCC(=O)OC(C)(C)C)c3ccc4ccccc4c3C2(C)C)C(C)(C)c2c1ccc1ccccc21 ; [Br-] ; COCCOCCOCCN1\C(=C/C=C/C=C/C=C/C2=[N+](CCOCCOCCOCCOCCOCCOCCOCCOCCOCCOCCOCCOCCC(=O)O)c3ccc4ccccc4c3C2(C)C)C(C)(C)c2c1ccc1ccccc21 ; [Br-]]

A solution of bis(indolium-m-dPEG₃-dPEG₁₂-TBE)-hepta-trienyl bromide (3.7 g, 2.71 mmol) in 30 mL of anhydrous dichloromethane was placed in a 100 mL three-neck round bottom flask equipped with a magnetic stirrer, thermocouple, nitrogen filled balloon, and cooling ice bath. The flask was covered with foil in order to protect from light, cooled to 5° C., and an excess of triethylsilane (1.46 g, 12.53 mmol) was added via syringe followed by the addition of trifluoroacetic acid (9.46 g, 30.6 mmol). The resulting orange solution was stirred at this temperature for 6 hours until all starting material was consumed, as determined by TLC. The reaction was concentrated under reduce pressure, and the obtained residue was diluted with dichloromethane (100 mL) and quenched with cold water (150 mL). The bottom organic layer was separated, aqueous phase was extracted with dichloromethane (3×80 mL), and the combined organic extracts were dried over anhydrous sodium sulfate. Drying agent was removed by filtration, and the filtrate was concentrated on rotavap to give 3.8 g of crude material as dark green viscous oil. The crude was purified by column chromatography on silica gel using gradient elution with dichloromethane-methanol mixture to give 2.66 g (75% yield) of product as viscous green oil.

¹H NMR (400 MHz, CDCl₃, δ): 8.12 (d, 2H, aromatic), 7.96-7.84 (m, 6H, aromatic/CH═CH), 7.60 (t, 2H, aromatic/CH═CH), 7.53-7.42 (m, 4H, aromatic), 6.57 (t, 2H), 6.29 (dd, 2H), 4.38 (dd, 4H), 3.94 (t, 4H), 3.81-3.43 (m, 58H, CH₂O), 3.40-3.35 (m, 2H), 3.27 (s, 3H, CH₃O), 2.67 (t, 2H, CH₂CO), 1.98 (s, 12H, CH₃)

### Example 5

### Bis-(m-dPEG₃-1,1-dimethyl-benzoinoliden)-hepta-1,3,5-trienyl-(dPEG₁₂-1,1-dimethyl-benzoindolium)-NHS ester

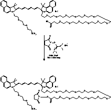

A solution of bis(indolium-m-dPEG₃-dPEG₁₂-acid)-hepta-trienyl bromide (2.61 g, 1.995 mmol) in 25 mL of anhydrous DMF was charged in a 100 mL three-neck round bottom flask equipped with a magnetic stirrer, thermocouple, nitrogen-filled balloon, and cooling ice bath. The flask was covered with foil in order to protect from light, cooled to 10° C., and TSTU tetrafluoroborate (1.05 g, 3.49 mmol) was added followed by the addition of DIEA (0.81 g, 6.27 mmol). Cooling bath was removed, and reaction stirred at ambient temperature for 5 hours until all starting material was consumed, as determined by TLC. The reaction was quenched with cold 10% HCl (2×100 mL), extracted with dichloromethane (3×80 mL), the organic layer was separated, aqueous phase extracted with dichloromethane (2×80 mL), and the combined organic extracts were dried over anhydrous sodium sulfate. Drying agent was removed by filtration, and the filtrate was concentrated on rotavap to give 2.9 g of crude material as dark green viscous oil. The crude was purified by column chromatography on silica gel using gradient elution with dichloromethane-isopropanol mixture to give 1.45 g (52% yield) of product as viscous green oil.

¹H NMR (400 MHz, CDCl₃, δ): 8.13 (d, 2H, aromatic), 7.99 (t, 2H), 7.93-7.86 (m, 4H, aromatic/CH═CH), 7.69 (1H, broad s), 7.59 (t, 2H, aromatic/CH═CH), 7.51-7.41 (m, 4H, aromatic), 6.52 (t, 2H), 6.25 (d, 2H), 4.34 (t, 4H), 3.94 (t, 4H), 3.87-3.45 (m, 58H, CH₂O), 3.41-3.35 (m, 2H), 3.29 (s, 3H, CH₃O), 2.89 (t, 2H, CH₂CO), 2.84 (s, 4H, succinimide), 1.99 (s, 12H, CH₃)

### Example 6

### 1,1,2-Trimethyl-benzindolinium butane sulfonate

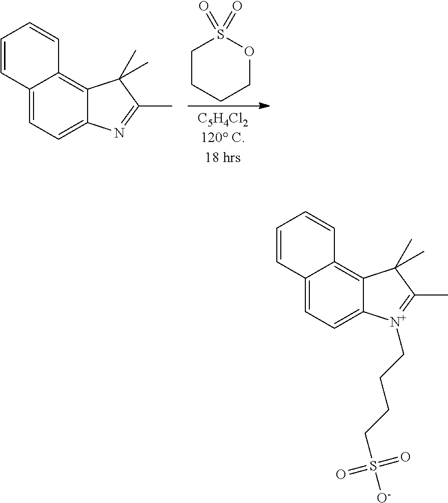

A total of 15 g (71.7 mmol) of 1,1,2-trimethylbenzindole and 287 mL of 1,2-dichlorobenzene was charged in a 500 mL three-neck round bottom flask equipped with a magnetic stirrer, thermocouple, nitrogen bleed, chilled condenser, and heating mantle. Neat 1,4-butane sulfone (22 mL, 29.3 g, 215 mmol) was added via syringe, and the reaction was heated at 120° C. for 18 hours in the dark until consumption of starting material by TLC in CH₂Cl₂/EtOH—HCO₂H=1:1. The dark purplish brown reaction was allowed to cool to room temperature, and the precipitate was collected on a Buchner funnel. The isolated solid was suspended in diethyl ether (100 mL) and filtered. The cake was washed again with ether (2×80 mL) and dried on a high vacuum pump for constant weight to afford 22.4 g (90% yield) of product as greenish gray solid.

¹H NMR (400 MHz, DMSO-d₆, δ): 8.37 (d, 1H, aromatic), 8.28 (d, 1H, aromatic), 8.22 (d, 1H, aromatic), 7.78 (t, 1H), 7.72 (t, 1H, aromatic), 4.62 (t, 2H, CH₂N), 2.96 (s, 3H, CH₃), 2.54 (t, 2H, CH₂—S), 2.04 (t, 2H, CH₂), 1.79 (m, 2H, CH₂), 1.76 (s, 6H, CH₃).

### Example 7

### 1,1,2-Trimethyl-N-phenylacetamido-hexa-1,3,5-trienyl-benzindolinium butane sulfonate

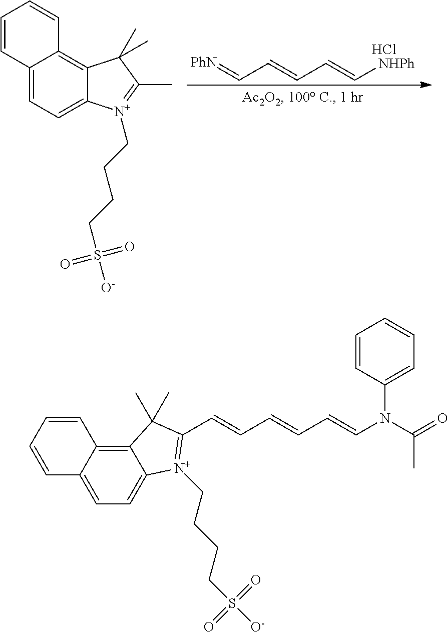

A mixture of 10 g (28.9 mmol) of 1,1,2-trimethylbenzindolinium butane sulfonate, 12.37 g (43.4 mmol) of glutaconaldehyde dianyl hydrochloride and 207 mL of acetic anhydride was charged in a 500 mL three-neck round bottom flask equipped with a magnetic stirrer, thermocouple, nitrogen bleed, chilled condenser, and heating mantle. The reaction was heated to 100° C. for 1 hour resulting in complete consumption of starting material by TLC in CH₂Cl₂/MeOH=9:1. The reaction mixture was cooled to ambient temperature and poured into 500 mL of cold (−20° C.) hexane resulting in separation of a dark oil. The hexane phase was decanted, and the residue dried on rotavap and the oil was taken up in 200 mL of ethyl acetate containing 2 mL of acetonitrile. The obtained mixture was poured into 600 mL of cold (−30° C.) ethyl acetate and the mixture vigorously stirred for 15 min. The formed dark purplish precipitate was collected on a Buchner funnel. The precipitate was suspended in 200 mL of hexane and filtered on a Buchner funnel again. The cake washed with hexane (100 mL) one more time and dried under high vacuum for 2 hours to give 18.5 g of crude material. This crude was purified by column chromatography on silica gel using gradient elution with dichloromethane-ethanol to give 13.8 g (88% yield) of product as a dark purplish solid.

¹H NMR (400 MHz, CD₃OD, δ): 8.27 (d, 1H, aromatic), 8.21-8.03 (m, 4H, aromatic), 7.86 (d, 1H, aromatic), 7.71 (t, 1H), 7.63-7.51 (m, 4H, aromatic/vinyl), 7.44-7.23 (m, 4H, aromatic/vinyl), 6.93 (d, 1H, vinyl), 6.56 (dd, 1H), 5.36 (dd, 1H), 4.50 (t, 2H, CH₂), 3.29 (s, 3H, CH₃CO), 2.84 (t, 2H, CH₂), 2.07 (m, 2H, CH₂), 2.96 (s, 3H, CH₃), 2.54 (t, 2H, CH₂—S), 2.04 (t, 2H, CH₂), 1.97-1.86 (m, 10H, CH₃, CH₂).

### Example 8

### 1,1,2-Trimethyl-benzindolinium-dPEG₁₂-TBE bromide

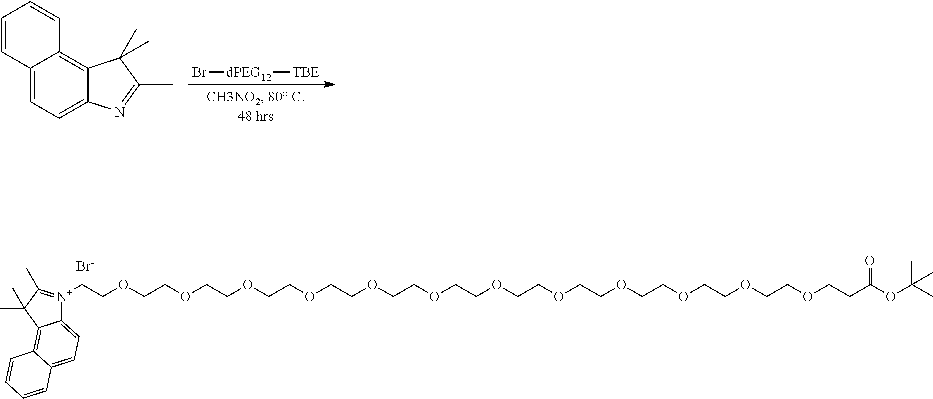

A solution of 16.76 g (22.71 mmol) of Br-dPEG₁₂-TBE in 45 mL of nitromethane was charged in a 250 mL three-neck round bottom flask equipped with a magnetic stirrer, thermocouple, nitrogen bleed, chilled condenser, and heating mantle. A total of 5.70 g (27.3 mmol) of 1,1,2-trimethylbenzindole was added in a single portion, and the reaction was heated to 80° C. for 72 hours resulting in an essential consumption of starting material, as determined by TLC in CH₂Cl₂/MeOH=95:5. The reaction mixture was cooled to ambient temperature and concentrated under reduced pressure and the residue was oiled out in hexane and the solvent decanted. This was repeated four times to remove as much unreacted starting materials as possible. The crude was purified by column chromatography on silica gel using gradient elution with dichloromethane/ethyl acetate=4/1-ethanol to give 7.12 g (33.1% yield) of product as a dark reddish blue oil.

¹H NMR (400 MHz, DMSO-d₆, δ): 8.38 (d, 1H, aromatic), 8.29 (d, 1H, aromatic), 8.21 (d, 1H, aromatic), 8.17 (d, 1H, aromatic), 7.79 (t, 1H), 7.73 (t, 1H), 4.87 (t, 2H, CH₂), 3.98 (m, 1H), 3.94 (t, 2H), 3.79 (t, 1H), 3.67-3.25 (m, 48H, CH₂O), 2.93 (s, 3H, CH₃), 2.40 (t, 2H, CH₂—CO), 1.77 (s, 6H, CH₃), 1.39 (s, 9H, t-Bu).

### Example 9

### dPEG₁₂-TBE-1,1-dimethyl-benzoindoliden)-hepta-1,3,5-trienyl-(1,1-dimethyl-benzoindolium butane sulfonate) (ICG-dPEG₁₂-TBE

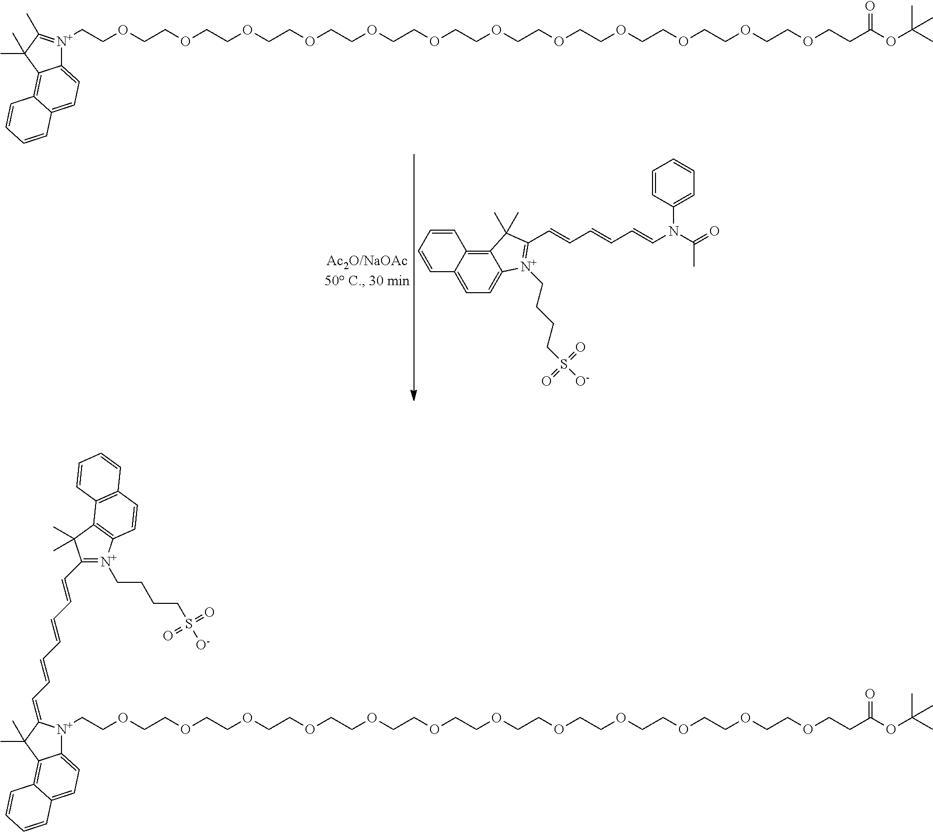

A solution of 1.8 g (3.32 mmol) of 1,1-dimethyl-N-phenylacetamido-hexa-1,3,5-trienyl-benzoindolium butane sulfonate in 33 mL of ethanol was charged in a 200 mL three-neck round bottom flask equipped with a magnetic stirrer, thermocouple, nitrogen bleed, chilled condenser, and heating mantle. A total of 0.327 g (3.98 mmol) of sodium acetate and 0.501 mL (5.31 mmol) of acetic anhydride were added followed by the addition of a solution of 3.46 g (3.65 mmol) of 1,1,2-trimethyl-benzindolinium-dPEG₁₂-TBE in 33 mL of ethanol. The reaction was heated to 50° C. and held for 30 minutes, resulting in the consumption of starting material by TLC in CH₂Cl₂/MeOH=9:1. The reaction mixture was cooled to ambient temperature and concentrated under reduced pressure. The crude material was purified by column chromatography on silica gel using gradient elution with dichloromethane/ethyl acetate=4/1-ethanol to give 3.375 g (80% yield) of product as a dark greenish blue solid.

¹H NMR (400 MHz, DMSO-d₆, δ): 8.25 (t, 2H, aromatic), 8.11-7.93 (m, 6H, aromatic/vinyl), 7.79 (d, 2H, aromatic), 7.72-7.59 (m, 3H, aromatic/vinyl), 7.54-7.44 (m, 2H, aromatic/vinyl), 6.67-6.48 (m, 3H, vinyl), 6.42 (d, 1H, aromatic/vinyl), 4.41 (t, 2H, CH₂), 4.24 (t, 2H), 3.83 (t, 2H), 3.60-3.27 (m, 48H, CH₂O), 2.55 (t, 2H), 2.40 (t, 2H, CH₂—CO), 1.92 (d, 6H, CH₃ and 8H CH₂), 1.39 (s, 9H, t-Bu).

### Example 10

### dPEG₁₂-acid-1,1-dimethyl-benzoindoliden)-hepta-1,3,5-trienyl-(1,1-dimethyl-benzoindolium butane sulfonate) (ICG-dPEG₁₂-CO2H

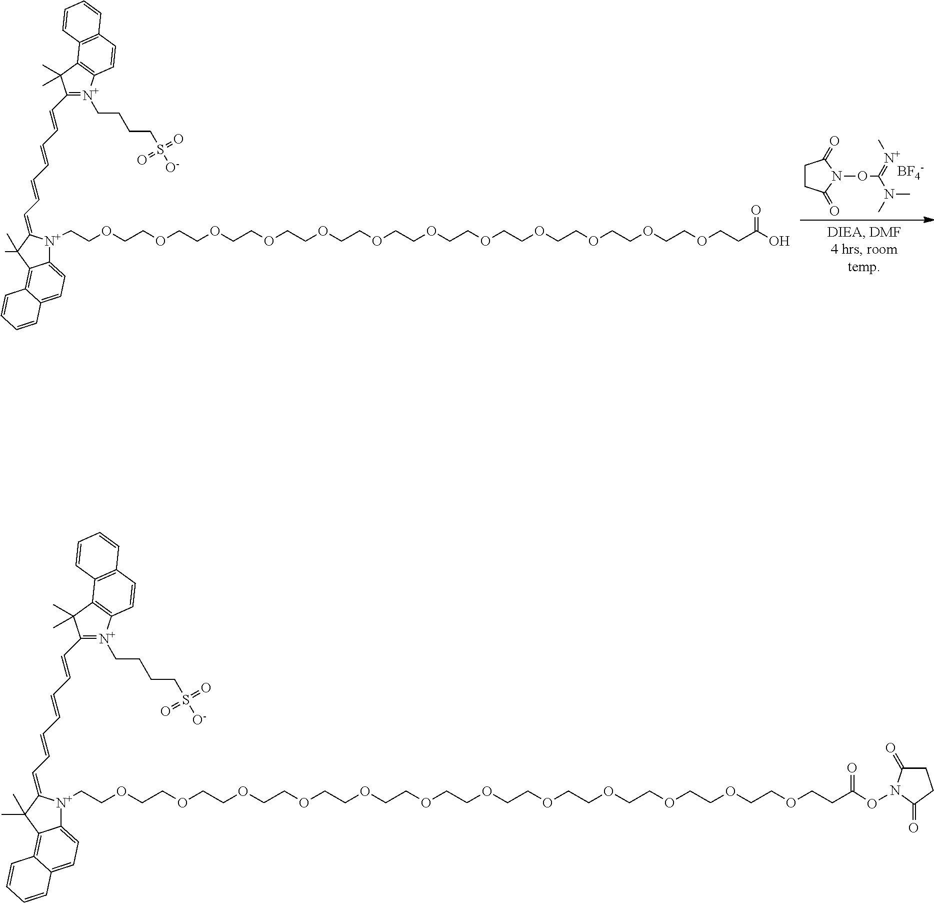

A solution of ICG-dPEG₁₂-TBE (3.375 g, 2.65 mmol) in 9 mL of anhydrous dichloromethane was placed in a 100 mL three-neck round bottom flask equipped with a magnetic stirrer, thermocouple, nitrogen filled balloon, and cooling ice bath. The flask was covered with foil to protect from light, cooled to 5° C., and an excess of triethylsilane (1.058 mL, 6.62 mmol) was added via syringe followed by the addition of trifluoroacetic acid (5.10 mL, 66.2 mmol). The cooling bath was removed, and the resulting orange solution was stirred at ambient temperature for five hours until all starting material was consumed by TLC in CH₂Cl₂/MeOH=9:1. The reaction was concentrated under reduced pressure to give dark green oil. The oil was suspended in hexane and the solvent decanted. This was repeated one more time, and the obtained oil was further purified by column chromatography on silica gel using gradient elution with dichloromethane-ethanol mixture to give 2.509 g (78% yield) of product as a dark green solid.

¹H NMR (400 MHz, DMSO-d₆, δ): 8.25 (t, 2H, aromatic), 8.09-7.92 (m, 6H, aromatic/vinyl), 7.79 (d, 2H, aromatic), 7.72-7.60 (m, 3H, aromatic/vinyl), 7.54-7.45 (m, 2H, aromatic/vinyl), 6.68-6.38 (m, 4H, vinyl), 4.41 (t, 2H, CH₂), 4.23 (t, 2H), 3.83 (t, 3H), 3.64-3.25 (m, 44H, CH₂O), 2.55 (t, 2H), 2.43 (t, 2H, CH₂—CO), 1.92 (d, 6H, CH₃), 1.91-1.73 (m, 8H, CH₂).

### Example 11

### dPEG₁₂-NHS ester)-1,1-dimethyl-benzoindoliden-hepta-1,3,5-trienyl-(1,1-dimethyl-benzoindolium butane sulfonate) (ICG-dPEG₁₂-NHS ester)

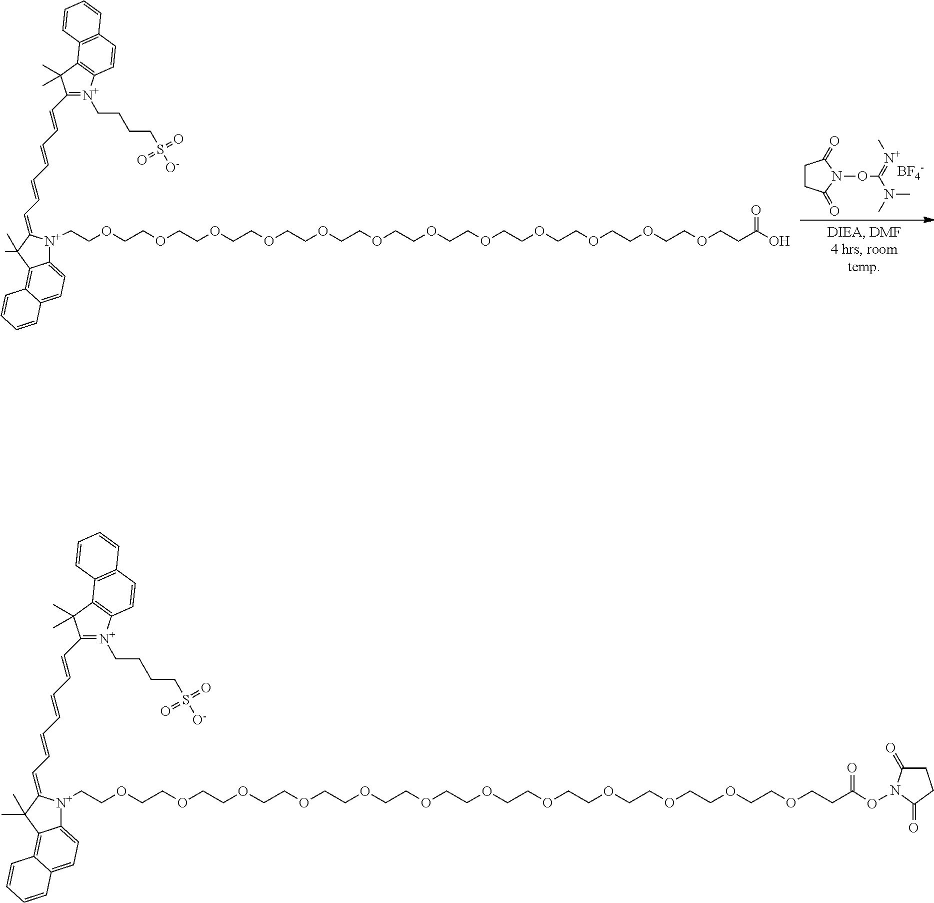

A solution of ICG-dPEG₁₂-acid (2.509 g, 2.061 mmol) in 21 mL of anhydrous DMF was charged in a 100 mL three-neck round bottom flask equipped with a magnetic stirrer, thermocouple, nitrogen filled balloon, and cooling ice bath. The flask was covered with foil to protect from light, cooled to 10° C., and TSTU tetrafluoroborate (0.735 g, 2.473 mmol) was added in a single portion followed by the addition of DIEA (0.468 mL, 2.68 mmol) via pipette. The cooling bath was removed, and the reaction stirred at ambient temperature for five hours until all of the starting material was consumed, as determined by TLC in dichloromethane-ethanol with 1% formic acid. The reaction was diluted with dichloromethane (150 mL), washed with cold 10% HCl (3×30 mL), washed with 1:1 brine/10% HCl (2×60 mL), and the organic phase was dried over anhydrous sodium sulfate. Drying agent was removed by filtration, and the filtrate was concentrated under reduced pressure to give crude material as dark green oily solid. The crude was taken up in 15 mL acetonitrile and dripped into 75 mL of hexane. The solvent was decanted as much as possible and the rest was chilled in a dry ice/acetone bath to help solidify the residue. The residual solvent was decanted and the oil dried under reduced pressure a sticky oily greenish-blue solid. The residue was repeatedly suspended in diethyl ether, crushed with a spatula, the solvent decanted and dried under high vacuum. This was repeated one more time until 2.18 g (80% yield) of a greenish-blue solid was obtained.

¹H NMR (400 MHz, DMSO-d₆, δ): 8.24 (t, 2H, aromatic), 8.12-7.91 (m, 6H, aromatic/vinyl), 7.79 (d, 2H, aromatic), 7.72-7.59 (m, 3H, aromatic/vinyl), 7.55-7.43 (m, 2H, aromatic/vinyl), 6.68-6.37 (m, 4H, vinyl), 4.41 (t, 2H, CH₂), 4.24 (t, 2H), 3.83 (t, 3H), 3.90-3.62 (m, 10H, CH₂O), 3.57-3.26 (m, 38H, CH₂O), 2.92 (t, 2H), 2.54 (t, 2H, CH₂—CO), 1.91 (s, 6H, CH₃), 1.91-1.71 (m, 8H, CH₂).

### Example 12

### Conjugation of Secondary Antibodies, IgG

### ICG-dPEG-Antibodies

The structure of ICG-dPEG₁₂-NHS is shown in FIG. 16 and the structure m-dPEG₃-ICG-dPEG₁₂-NHS is shown in FIG. 17. The maximal number of ICGs using this reagent that can be incorporated into an IgG without losing the protein is 4-5 ICG/IgG (FIG. 18). A similar number of ICGs from ICG-NHS, the reagent without dPEG, can be substituted into IgG (FIG. 19). An ELISA assay of ICG-MAG (FIG. 20) showed that antibody binding activity was not lost after ICG-dPEG or ICG was incorporated.

The fluorescence of the different ICG constructs was measured using an excitation wavelength of 780 nm and an emission wavelength of 810 nm. We found that both ICG-dPEG₁₂-IgG and m-dPEG₃-ICG-dPEG₁₂-ICG fluoresced at ˜3× the intensity of ICG-IgG (no dPEG). In these experiments, all of the ICG conjugates were prepared the same day as the fluorescence readings were made since ICG is intrinsically unstable in aqueous solution.

ICG is a hydrophobic molecule that forms a tight complex with Human Serum Albumin (HSA). The fluorescence of ICG increased ˜10 fold, as expected, when it was added to a 4% HSA solution. When ICG is conjugated to a protein, it loses its ability to form tight HAS complexes, as confirmed in our experiments where the fluorescence of ICG-IgG (no dPEG) increased by only a factor of ˜2 when added to 4% HSA. We hypothesized that ICG might still be able to bind to HSA when it was bound to IgG through a dPEG₁₂ linker. However, when we added ICG-dPEG₁₂-IgG to 4% HAS, the fluorescence increased by a factor of ˜2, the same increase as found for the conjugate without the dPEG linker, indicating that ICG in the dPEG conjugate does not bind tightly to HSA.

a. Structure of ICG-dPEG₁₂-NHS

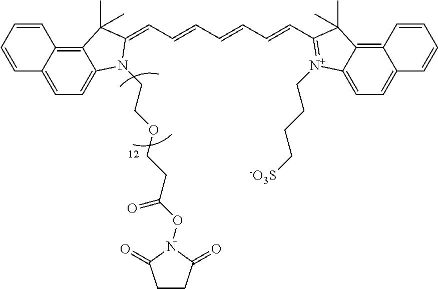

b. Structure of dPEG₃-ICG-dPEG₁₂-NHS

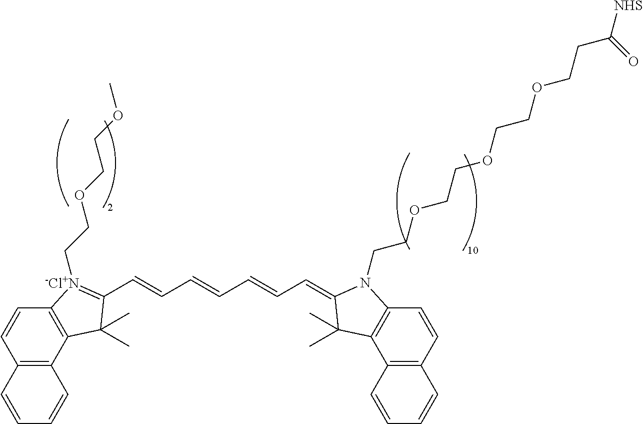

c. Conjugation of ICG-dPEG₁₂-NHS with MAG (Mouse Anti-Goat IgG Antibody)

MAG reacted with different concentrations of ICG-dPEG₁₂-NHS in 10% DMAC

Product purified over two G50 spin columns in PBS

[IgG] and ICG/IgG molar ratios determined from A (770 nm) and A (280) nm

Lose most of protein when [ICG-dPEG₁₂-NHS] in the reaction is ≧0.2 mM

Highest incorporation: ICG/IgG˜5

When ICG/IgG˜5, approximately 50% of the conjugate precipitates on ON storage at 4° C.

In a sample where ICG/IgG 4, no conjugate was lost on ON storage

d.e. Conjugation of ICG-NHS (no dPEG) with MAG

Soluble in 10% DMSO (lit) or DMAC (our work)

Reacted with MAG but couldn't separate unreacted reagent on spin columns in PBS

Unreacted reagent (no MAG control) apparently forms aggregates that are partially excluded from spin columns

Can purify on a PD50 column in 20% DMAC followed by a PD 10 column in PBS.

Obtained a conjugate with ICG/IgG˜5

### Example 13

### Competition ELISA for ICG-dPEG-MAG (MAG-d-ICG) and ICG-MAG (MAG-ICG)

FIG. 1 provides the results.

### Example 14

### CF(5,6)-dPEG₁₂-NHS conjugates with proteins

The data shown below shows the very unexpected performance characteristics for the CF(5,6)-dPEG₁₂-NHS when conjugated to a larger protein, e.g., an IgG antibody or to streptavidin. Primarily we show that we can put on almost 30 molecules of CF(5,6)-dPEG₁₂-NHS per protein molecule without any self-quenching, while carboxy fluorescein, or the FITC equivalent which is a standard in the industry, one can put generally only 3 to 4 per protein before self-quenching limits the intensity. We also find that the stability of the CF(5,6)-dPEG₁₂-protein conjugates are very unexpectedly photostable when compared to FITC, which is very photolabile and the Alexa-488, which is very stable.
a. High dye loading without self-quenching.

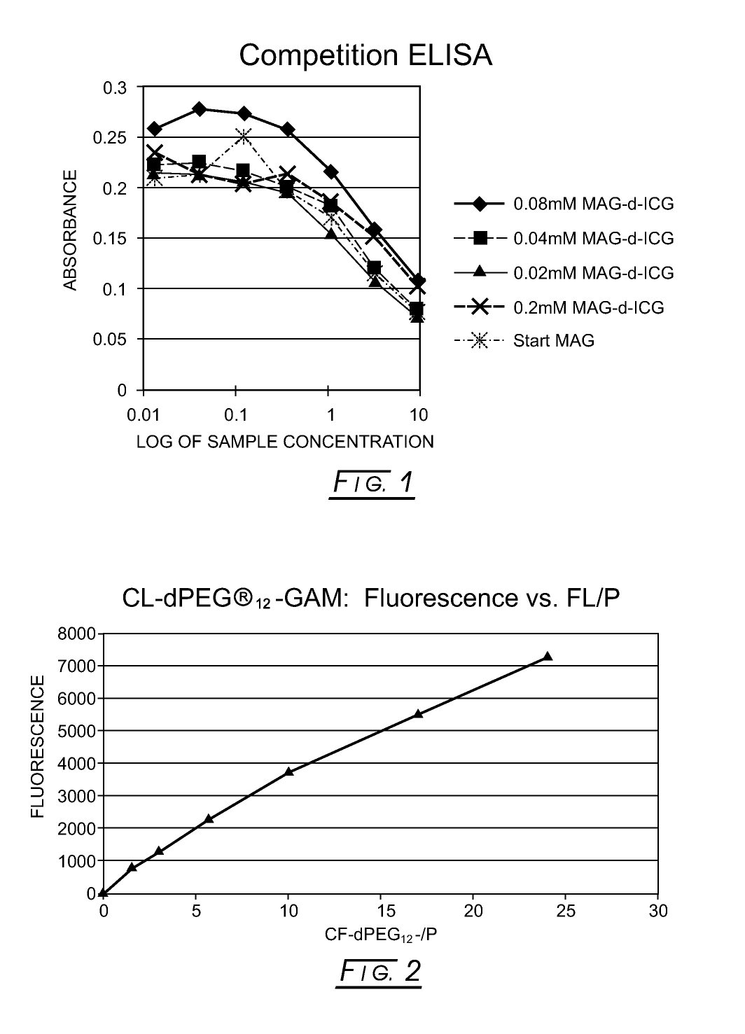

The results are displayed in FIG. 2.
b. Comparing the intensity and performance of the Goat anti-mouse (GAM) CF-dPEG₁₂-conjugates against Alexa-488 and FITC conjugates

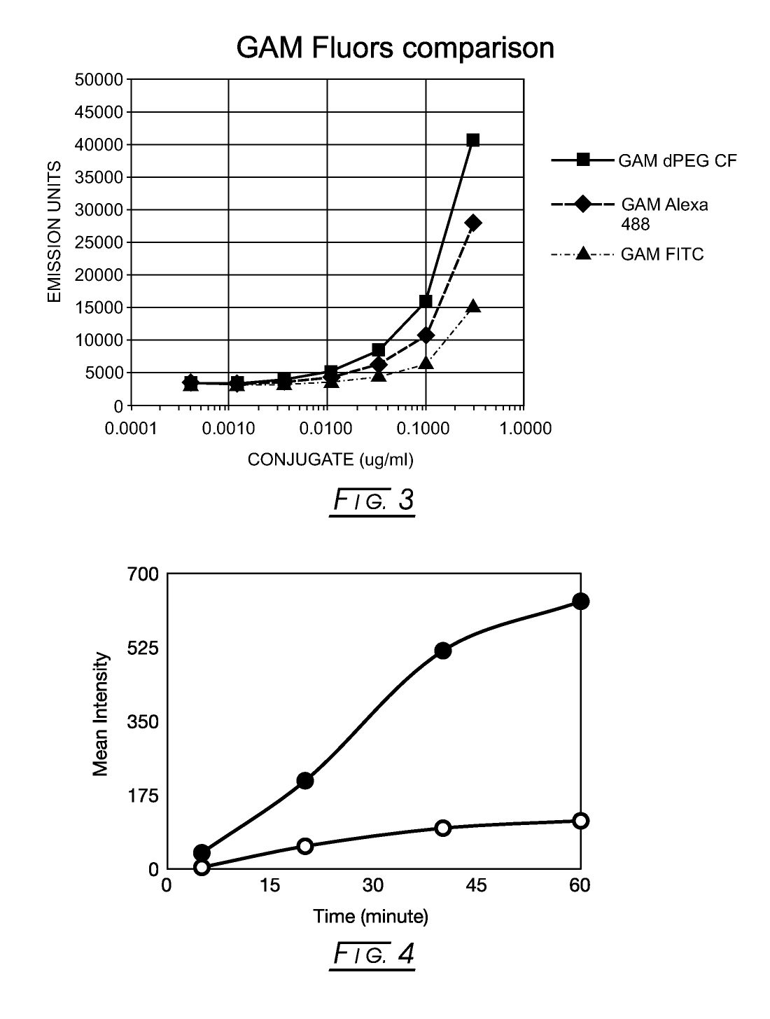

A Dark ELISA plate was coated with Mouse IgG at a constant concentration of [0.3 μg/ml]. Conjugates diluted in PBS-Tween to a highest concentration of [0.4 μg,L]. then diluted by ⅓ from row to row on the plate. The resulting fluorescence was measured on a Tecan plate reader. The results are displayed in FIG. 3.
c. Photostability of conjugates. Each conjugate in solution was scanned 40 times (each scan would be typical of what a conjugate would be subjected to in a standard assay. The GAM-Alexa-488's fluorescent intensity decreased by 2.5% after 40 scans, the GAM-dPEG₁₂-CF decreased by 4.0% and the FITC was diminished to less than 25% of the original (as the standard expectation for fluorescein dyes).
d. Effect of linker on the cell internalization, CF-dPEG₁₂- vs. FITC. In the figure below it can be seen that effect of the dPEG₁₂ linker attached to the peptide vs. having no linker is almost a factor of 10 fold, a dramatic effect indeed. The cells used are liver cells and the peptide was designed to target HCV in the cell. The upper line is for the peptide with the CF-dPEG₁₂ attached to the N-terminus and the lower line when FITC is attached to the peptide. The time scale is for the time the peptide is incubated with the cells. The cells are washed, lysed, and the peptide-dye internalized is measured. The results are displayed in FIG. 4. Shown below is the structure for the 5-isomer of the CF-dPEG₁₂ NHS ester, of the 5(6) mixture of isomers used to derivatize the peptide used in the current example.

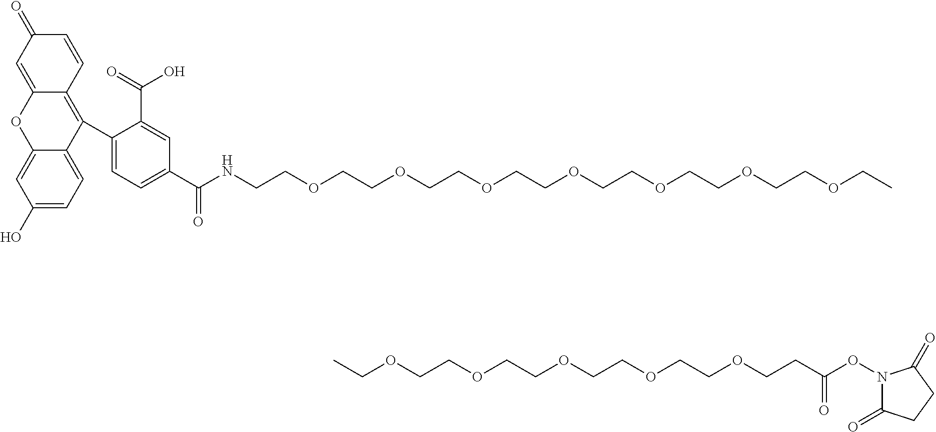

While the compositions and methods have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

I claim:

1. Modified dyes comprising a dye substituted with: 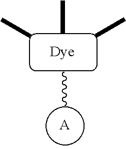
where:
(a) (A) is a reactive or reactable group;
(b) the solid line, [symbol] , is one or more linear discrete PEG-containing chain having between about 1 and 64 ethylene oxide residues and having a terminal moiety that is not an ethylene oxide residue; and
(c) the wavy line, [symbol] is a linear discrete PEG containing chain optionally substituted with one or more of N, S, Si, Se, or P and terminated with a reactive or a reactable group (A).

2. The modified dye of claim 1, wherein A is one or more of a cell surface and matrix antigen, transport protein, receptor protein, an antibody, antibody fragment, engineered antibody, engineered fragment, peptide, peptide substrate, peptomimetic substrate, cytokine, aptamer, siRNA, vitamin, steroid, a nanoparticle, microparticle, engineered molecular scaffold that contains multiple functionalities, or an engineered chemically reactable group.

3. The modified dye of claim 1, wherein A is a chemically moiety reactable with thiols, carbonyls, carboxylic acid, amines, strained alkenes, strained alkynes, or azides.

4. The modified dye of claim 1, wherein the solid line, [symbol] , is terminated by a charged group that is either positive or zwitterionic.

5. The modified dye of claim 1, wherein each wavy line, [symbol] , contains between about 2 and 24 ethylene oxide groups.

6. The modified dye of claim 1, wherein each solid line, [symbol] , independently, contains between about 3 and 24 ethylene oxide groups.

7. Modified dyes of the cyanine class substituted with:

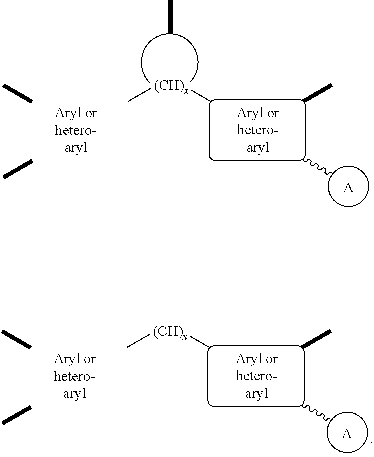

8. Modified dyes and their intermediates being one or more of:
(a) m-dPEGₓ-1,1,2-trimethyl-benzoindolium bromide; x=3 to 24, and x to 24-amido-24;
(b) m-dPEGₓ-1,1-dimethyl-N-phenylacetamido-hexa-1,3,5-trienyl-benzoindolium bromide; x=3 to 24, and x to 24-amido-24;
(c) Bis-(m-dPEGₓ-1,1-dimethyl-benzoinoliden)-hepta-1,3,5-trienyl-(dPEGₓ-t-butyl ester-1,1-dimethyl-benzoindolium) bromide; x=3 to 24, and x to 24-amido-24;
(d) Bis-(m-dPEGₓ-1,1-dimethyl-benzoinoliden)-hepta-1,3,5-trienyl-(dPEGₓ-acid-1,1-dimethyl-benzoindolium) bromide; x=3 to 24, and x to 24-amido-24;
(e) Bis-(m-dPEGₓ-1,1-dimethyl-benzoinoliden)-hepta-1,3,5-trienyl-(dPEGₓ-yl-1,1-dimethyl-benzoindolium) NHS ester; x=3 to 24, and x to 24-amido-24;
(f) 1,1,2-Trimethyl-benzindolinium-dPEGₓ-t-butyl ester bromide; x=3 to 24, and x to 24-amido-24;
(g) 5(6)-Carboxyfluorescein-dPEGₓ-NHS ester; x=3 to 24, and x to 24-amido-24; or
(h) 5(6)-Carboxyfluorescein-dPEG₁₂-NHS ester.
